(19) United States
(12) Patent Application Publication
(10) Pub. No.: US 2025/0356952 A1
(43) Pub. Date: Nov. 20, 2025

(54) FRAGMENTOMICS FOR ESTIMATING FETAL FRACTION IN NON-INVASIVE PRENATAL TESTING

(71) Applicant: Illumina, Inc., San Diego, CA (US)

(72) Inventors: Fan SONG, San Diego, CA (US); Cosmin DECIU, San Diego, CA (US); Mahdi GOLKARAM, San Diego, CA (US); Mike MEHAN, San Diego, CA (US); Chen ZHAO, San Diego, CA (US)

(21) Appl. No.: 18/871,442

(22) PCT Filed: Mar. 7, 2024
(86) PCT No.: PCT/US2024/018829

§ 371 Date: Dec. 3, 2024

Related U.S. Application Data
(60) Provisional application No. 63/451,151, filed on Mar. 9, 2023.

(51) Int. Cl. G16B 30/10; G16B 40/20

(52) U.S. Cl. CPC G16B 30/10; G16B 40/20

(57) ABSTRACT

The technology relates in part to estimating fetal fraction in non-invasive prenatal testing using one or more fragmentomics parameters. In some aspects, the technology relates to estimating fetal fraction according to nucleic acid fragment lengths and sequence motif frequencies.

### RELATED PATENT APPLICATION(S)

This patent application is a 35 U.S.C. 371 national phase application of International Patent Cooperation Treaty (PCT) Application No. PCT/US2024/018829, filed on Mar. 7, 2024, entitled FRAGMENTOMICS FOR ESTIMATING FETAL FRACTION IN NON-INVASIVE PRENATAL TESTING, naming Fan SONG et al. as inventors, and designated by Attorney Docket No. ILM-1001PCT, which claims the benefit of U.S. provisional patent application No. 63/451,151 filed on Mar. 9, 2023, entitled FRAGMENTOMICS FOR ESTIMATING FETAL FRACTION IN NON-INVASIVE PRENATAL TESTING, naming Fan SONG et al. as inventors, and designated by Attorney Docket No. ILM-1001PROV. The entire content of the foregoing patent application is incorporated herein by reference for all purposes, including all text, tables and drawings.

### FIELD

The technology relates in part to estimating fetal fraction in non-invasive prenatal testing using one or more fragmentomics parameters. In some aspects, the technology relates to estimating fetal fraction according to nucleic acid fragment lengths and sequence motif frequencies.

### BACKGROUND

Genetic information of living organisms (e.g., animals, plants and microorganisms) and other forms of replicating genetic information (e.g., viruses) is encoded in deoxyribonucleic acid (DNA) or ribonucleic acid (RNA). Genetic information is a succession of nucleotides or modified nucleotides representing the primary structure of chemical or hypothetical nucleic acids. In humans, the complete genome contains about 30,000 genes located on twenty-three (23) chromosomes. Each gene encodes a specific protein, which after expression via transcription and translation fulfills a specific biochemical function within a living cell.

Many medical conditions are caused by one or more genetic variations. Certain genetic variations cause medical conditions that include, for example, hemophilia, thalassemia, Duchenne Muscular Dystrophy (DMD), Huntington's Disease (HD), Alzheimer's Disease and Cystic Fibrosis (CF). Such genetic diseases can result from an addition, substitution, or deletion of a single nucleotide in DNA of a particular gene. Certain birth defects are caused by a chromosomal abnormality, also referred to as an aneuploidy, such as Trisomy 21 (Down's Syndrome), Trisomy 13 (Patau Syndrome), Trisomy 18 (Edward's Syndrome), Monosomy X (Turner's Syndrome) and certain sex chromosome aneuploidies such as Klinefelter's Syndrome (XXY), for example. Another genetic variation is fetal gender, which can often be determined based on sex chromosomes X and Y. Some genetic variations may predispose an individual to, or cause, any of a number of diseases such as, for example, diabetes, arteriosclerosis, obesity, various autoimmune diseases and cancer (e.g., colorectal, breast, ovarian, lung).

Identifying one or more genetic variations or variances can lead to diagnosis of, or determining predisposition to, a particular medical condition. Identifying a genetic variance can result in facilitating a medical decision and/or employing a helpful medical procedure. In certain embodiments, identification of one or more genetic variations or variances involves the analysis of cell-free DNA. Cell-free DNA (cfDNA) is composed of DNA fragments that originate from cell death and circulate in peripheral blood. High concentrations of cfDNA can be indicative of certain clinical conditions such as cancer, trauma, burns, myocardial infarction, stroke, sepsis, infection, and other illnesses. Additionally, cell-free fetal DNA (cffDNA) can be detected in the maternal bloodstream and used for various noninvasive prenatal diagnostics.

The presence of fetal nucleic acid in maternal plasma allows for non-invasive prenatal diagnosis through the analysis of a maternal blood sample. For example, quantitative abnormalities of fetal DNA in maternal plasma can be associated with a number of pregnancy-associated disorders, including preeclampsia, preterm labor, antepartum hemorrhage, invasive placentation, fetal Down syndrome, and other fetal chromosomal aneuploidies. Hence, fetal nucleic acid analysis in maternal plasma can be a useful mechanism for the monitoring of feto-maternal well-being.

Fetal fraction (FF) is the percentage of maternal plasma cell free DNA (cfDNA) that is of fetoplacental origin. Accurate measurement of FF is critical to non-invasive prenatal testing (NIPT) quality control and performance. Low FF can result in a “no call” result due to limit of detection (LOD). Higher FF leads to a greater statistical separation of aneuploid and euploid pregnancies, and increases detection rates. Described herein is a method that utilizes nucleic acid fragment lengths and fragment end motif frequencies in a machine learning framework for FF estimation.

### SUMMARY

Provided in certain aspects are methods for estimating a fraction of fetal nucleic acid in a test sample from a pregnant subject comprising a) obtaining sequence reads mapped to a reference genome, where the sequence reads are reads of circulating cell-free (CCF) nucleic acid from a test sample from a pregnant subject; b) measuring fragment lengths for a plurality of circulating cell-free nucleic acid fragments; c) generating one or more fragment length profiles for the test sample; d) determining a sequence motif for a plurality of circulating cell-free nucleic acid fragment ends; e) determining one or more sequence motif frequencies for the test sample; and f) estimating a fraction of fetal nucleic acid for the test sample according to i) the one or more fragment length profiles for the test sample, and ii) the one or more sequence motif frequencies for the test sample.

Also provided in certain aspects are systems comprising one or more microprocessors and memory, which memory comprises instructions executable by the one or more microprocessors and which memory comprises sequence reads mapped to a reference genome, which sequence reads are reads of circulating cell-free (CCF) nucleic acid from a test sample from a pregnant subject, and which instructions executable by the one or more microprocessors are configured to a) measure fragment lengths for a plurality of circulating cell-free nucleic acid fragments; b) generate one or more fragment length profiles for the test sample; c) determine a sequence motif for a plurality of circulating cell-free nucleic acid fragment ends; d) determine one or more sequence motif frequencies for the test sample; and e) estimate a fraction of fetal nucleic acid for the test sample according to i) the one or more fragment length profiles for the test sample, and ii) the one or more sequence motif frequencies for the test sample.

Also provided in certain aspects are machines comprising one or more microprocessors and memory, which memory comprises instructions executable by the one or more microprocessors and which memory comprises sequence reads mapped to a reference genome, which sequence reads are reads of circulating cell-free nucleic acid from a test sample from a pregnant subject, and which instructions executable by the one or more microprocessors are configured to a) measure fragment lengths for a plurality of circulating cell-free nucleic acid fragments; b) generate one or more fragment length profiles for the test sample; c) determine a sequence motif for a plurality of circulating cell-free nucleic acid fragment ends; d) determine one or more sequence motif frequencies for the test sample; and e) estimate a fraction of fetal nucleic acid for the test sample according to i) the one or more fragment length profiles for the test sample, and ii) the one or more sequence motif frequencies for the test sample.

Also provided in certain aspects are non-transitory computer-readable storage media with an executable program stored thereon, where the program instructs a microprocessor to perform the following: a) access sequence reads mapped to a reference genome, which sequence reads are reads of circulating cell-free nucleic acid from a test sample from a pregnant subject, b) measure fragment lengths for a plurality of circulating cell-free nucleic acid fragments; c) generate one or more fragment length profiles for the test sample; d) determine a sequence motif for a plurality of circulating cell-free nucleic acid fragment ends; e) determine one or more sequence motif frequencies for the test sample; and f) estimate a fraction of fetal nucleic acid for the test sample according to i) the one or more fragment length profiles for the test sample, and ii) the one or more sequence motif frequencies for the test sample.

Certain implementations are described further in the following description, examples and claims, and in the drawings.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1 shows an example workflow of a method described herein. First panel: data collection—an internal dataset generated from cfDNA sequencing for ˜1500 pregnant women. Second panel: feature extraction and data processing. Sequencing data were processed with Illumina's DRAGEN platform to obtain fragment profile at 5 Mb genomic bins and sequence motif frequencies at chromosome levels. Third panel: dimension reduction—features were analyzed with principal component analysis (PCA) to extract the most relevant variables that predict fetal fraction. The dataset was then split into training and testing at an 80-20 ratio. Fourth panel: model training—three models including linear regression, Elastic net, and XGBoost were used in model training. Techniques such as randomized parameter search and 5-fold cross-validation (CV) were used to prevent overfitting of the model. Fifth panel: model was evaluated on the test data set using two metrics root mean squared error (RMSE) and correlation with truth fetal fraction.

FIG. 2 shows the DRAGEN data processing method is at least two orders of magnitude faster than existing tools.

FIG. 3 shows fragment profile (upper row) and sequence motif frequency (bottom row) from DRAGEN (x-axis) is highly concordant with existing tool (y-axis) in TSO500 and whole exome sequencing (WES) cfDNA datasets.

FIG. 4 shows models trained with only fragment size and Elastic net or XGBoost models. Fetal fraction predicted by model (y-axis) showed high consistency with truth data (x-axis).

FIG. 5 shows models trained with only 5′-end sequence motif frequencies and Elastic net or XGBoost models. Fetal fraction predicted by model (y-axis) showed high consistency with truth data (x-axis).

FIG. 6 provides a table showing a sequence motif-based method showed similar error rate of 2.5% when compared to a traditional fragment size-based method.

FIG. 7 shows an overview of performance on a variety of models, which indicates combining sequence motif with fragment size or coverage features improves prediction accuracy. RMSE (left) and correlation (right) of different models. Y-axis from top to bottom are predictions from 1) distributed random forest (DRF) using only sequence motif; 2) gradient boosting (GBM) using only sequence motif; 3) XGBoost using only sequence motif; 4) FF_Size model from NIPT team; 5) Elastic net/Generalized linear model (GLM) using only sequence motif; 6) FF_Coverage model which utilizes genome-wide read coverage to predict fetal fraction; 7) GLM model using both fragment size and sequence motif; 8) FF_Cov_Size model from NIPT team; 9) GLM model using fragment coverage and sequence motif; 10) FF4 model from NIPT team; 11) XGBoost model using fragment size, coverage, and sequence motif; 12) FF_Cov_Size_recompute model from NIPT team; 13) GLM model using fragment size, coverage, and sequence motif.

### DETAILED DESCRIPTION

Provided herein are methods and systems for estimating a fraction of fetal nucleic acid in a test sample. The systems and methods herein may include estimating a fraction of fetal nucleic acid for a test sample according to i) the one or more fragment length profiles for the test sample, and ii) the one or more sequence motif frequencies for the test sample.

### Determining Fetal Nucleic Acid Content

Methods and systems herein are directed estimating the amount of fetal nucleic acid (e.g., concentration, relative amount, absolute amount, copy number, and the like) in nucleic acid. In certain embodiments, the amount of fetal nucleic acid in a sample (e.g., test sample) is referred to as “fraction of fetal nucleic acid” or “fetal fraction.” In some embodiments, “fetal fraction” refers to the fraction of fetal nucleic acid in circulating cell-free nucleic acid in a sample (e.g., a blood sample, a serum sample, a plasma sample) obtained from a pregnant subject. Fetal fraction may be estimated according to fragment length profiles and sequence motif frequencies as described herein. Fetal fraction may be estimated by applying one or more model parameters to fragment length profiles and sequence motif frequencies determined for a test sample, as described herein. Model parameters may be obtained from a training set of samples for which fetal fraction in known (e.g., samples premixed with known amounts of maternal and fetal nucleic acid or samples for which fetal fraction is determined according to any suitable method in the art). Determining fetal fraction for training samples and/or test samples (e.g., for assessing accuracy of a fetal fraction estimation method herein) can be performed in a suitable manner, non-limiting examples of which include methods described below.

In certain embodiments, the amount of fetal nucleic acid is determined according to markers specific to a male fetus (e.g., Y-chromosome STR markers (e.g., DYS 19, DYS 385, DYS 392 markers); RhD marker in RhD-negative females), allelic ratios of polymorphic sequences, or according to one or more markers specific to fetal nucleic acid and not maternal nucleic acid (e.g., differential epigenetic biomarkers (e.g., methylation; described in further detail below) between mother and fetus, or fetal RNA markers in maternal blood plasma.

Determination of fetal nucleic acid content (e.g., fetal fraction) sometimes is performed using a fetal quantifier assay (FQA). This type of assay allows for the detection and quantification of fetal nucleic acid in a maternal sample based on the methylation status of the nucleic acid in the sample. In certain embodiments, the amount of fetal nucleic acid from a maternal sample can be determined relative to the total amount of nucleic acid present, thereby providing the percentage of fetal nucleic acid in the sample. Methods for differentiating nucleic acid based on methylation status include, but are not limited to, methylation sensitive capture, for example, using a MBD2-Fc fragment in which the methyl binding domain of MBD2 is fused to the Fc fragment of an antibody (MBD-FC); methylation specific antibodies; bisulfite conversion methods, for example, MSP (methylation-sensitive PCR), COBRA, methylation-sensitive single nucleotide primer extension (Ms-SNuPE) or Sequenom MassCLEAVE™ technology; and the use of methylation sensitive restriction enzymes (e.g., digestion of maternal DNA in a maternal sample using one or more methylation sensitive restriction enzymes thereby enriching the fetal DNA). Methyl-sensitive enzymes also can be used to differentiate nucleic acid based on methylation status, which, for example, can preferentially or substantially cleave or digest at their DNA recognition sequence if the latter is non-methylated. Thus, an unmethylated DNA sample will be cut into smaller fragments than a methylated DNA sample and a hypermethylated DNA sample will not be cleaved.

In certain embodiments, fetal fraction can be determined based on allelic ratios of polymorphic sequences (e.g., single nucleotide polymorphisms (SNPs). In such a method, nucleotide sequence reads are obtained for a maternal sample and fetal fraction is determined by comparing the total number of nucleotide sequence reads that map to a first allele and the total number of nucleotide sequence reads that map to a second allele at an informative polymorphic site (e.g., SNP) in a reference genome. In certain embodiments, fetal alleles are identified, for example, by their relative minor contribution to the mixture of fetal and maternal nucleic acids in the sample when compared to the major contribution to the mixture by the maternal nucleic acids. Accordingly, the relative abundance of fetal nucleic acid in a maternal sample can be determined as a parameter of the total number of unique sequence reads mapped to a target nucleic acid sequence on a reference genome for each of the two alleles of a polymorphic site.

### Samples

Provided herein are methods and compositions for analyzing nucleic acid. In some embodiments, nucleic acid fragments in a mixture of nucleic acid fragments are analyzed. A mixture of nucleic acids can comprise two or more nucleic acid fragment species having different nucleotide sequences, different fragment lengths, different origins (e.g., genomic origins, fetal vs. maternal origins, tumor vs. host origins, cell or tissue origins, sample origins, subject origins, and the like), or combinations thereof.

Nucleic acid or a nucleic acid mixture utilized in methods and apparatuses described herein often is isolated from a sample obtained from a subject. A subject can be any living or non-living organism, including but not limited to a human, a non-human animal, a plant, a bacterium, a fungus or a protist. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male, female, intersex, or non-binary. A subject may be any age (e.g., an embryo, a fetus, infant, child, adult). A subject may be pregnant or non-pregnant.

Nucleic acid may be isolated from any type of suitable biological specimen or sample (e.g., a test sample). A sample or test sample can be any specimen that is isolated or obtained from a subject or part thereof (e.g., a human subject, a pregnant subject, a fetus). Non-limiting examples of specimens include fluid or tissue from a subject, including, without limitation, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo), celocentesis sample, cells (blood cells, tumor cells, placental cells, embryo or fetal cells, fetal nucleated cells, fetal cellular remnants) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a biological sample is a cervical swab from a subject. In some embodiments, a biological sample is blood. The term “blood” as used herein refers to a blood sample or preparation from a pregnant subject or a subject being tested for possible pregnancy. The term encompasses whole blood, blood product or any fraction of blood, such as serum, plasma, buffy coat, or the like as conventionally defined. Blood or fractions thereof often comprise nucleosomes (e.g., maternal and/or fetal nucleosomes). Nucleosomes comprise nucleic acids and are sometimes cell-free or intracellular. Blood also comprises buffy coats. Buffy coats are sometimes isolated by utilizing a ficoll gradient. Buffy coats can comprise white blood cells (e.g., leukocytes, T-cells, B-cells, platelets, and the like). In certain embodiments, buffy coats comprise maternal and/or fetal nucleic acid. In some embodiments, a biological sample is blood plasma. Blood plasma refers to the fraction of whole blood resulting from centrifugation of blood treated with anticoagulants. In some embodiments, a biological sample is blood serum. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to or after preparation. A fluid or tissue sample from which nucleic acid is extracted may be acellular (e.g., cell-free). In some embodiments, a fluid or tissue sample may contain cellular elements or cellular remnants. In some embodiments, fetal cells or cancer cells may be included in the sample.

A sample often is heterogeneous, by which is meant that more than one type of nucleic acid species is present in the sample. For example, heterogeneous nucleic acid can include, but is not limited to, (i) fetal derived and maternal derived nucleic acid, (ii) cancer and non-cancer nucleic acid, (iii) pathogen and host nucleic acid, and more generally, (iv) mutated and wild-type nucleic acid. A sample may be heterogeneous because more than one cell type is present, such as a fetal cell and a maternal cell, a cancer and non-cancer cell, or a pathogenic and host cell. In some embodiments, a minority nucleic acid species and a majority nucleic acid species is present.

For prenatal applications of the technology described herein, a fluid or tissue sample may be collected from a subject at a gestational age suitable for testing, or from a subject who is being tested for possible pregnancy. Suitable gestational age may vary depending on the prenatal test being performed. A pregnant subject may be in the first trimester of pregnancy, may be in the second trimester of pregnancy, or may be in the third trimester of pregnancy. In certain embodiments, a fluid or tissue is collected from a pregnant subject between about 1 to about 45 weeks of fetal gestation (e.g., at 1-4, 4-8, 8-12, 12-16, 16-20, 20-24, 24-28, 28-32, 32-36, 36-40 or 40-44 weeks of fetal gestation), and sometimes between about 5 to about 28 weeks of fetal gestation (e.g., at 6, 7, 8, 9,10, 11, 12, 13, 14, 15, 16, 17, 18, 19, 20, 21, 22, 23, 24, 25, 26 or 27 weeks of fetal gestation). In certain embodiments, a fluid or tissue sample is collected from a pregnant subject during or just after (e.g., 0 to 72 hours after) giving birth (e.g., vaginal or non-vaginal birth (e.g., surgical delivery)).

### Acquisition of Samples and Extraction of Nucleic Acid

Methods herein may include separating, enriching and analyzing fetal DNA found in maternal blood as a non-invasive means to detect the presence or absence of a maternal and/or fetal genetic variation and/or to monitor the health of a fetus and/or a pregnant subject during and sometimes after pregnancy. Thus, the first steps of practicing certain methods herein may include obtaining a blood sample from a pregnant subject and extracting DNA from a sample.

A blood sample can be obtained from a pregnant subject at a gestational age suitable for testing using a method of the present technology. A suitable gestational age may vary depending on the disorder tested. Collection of blood from a subject often is performed in accordance with the standard protocol hospitals or clinics generally follow. An appropriate amount of peripheral blood, e.g., typically between 5-50 ml, often is collected and may be stored according to standard procedure prior to further preparation. Blood samples may be collected, stored or transported in a manner that minimizes degradation or the quality of nucleic acid present in the sample.

An analysis of fetal DNA found in maternal blood may be performed using, e.g., whole blood, serum, or plasma. Methods for preparing serum or plasma from maternal blood are known. For example, a pregnant subject's blood can be placed in a tube containing EDTA or a specialized commercial product such as Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for DNA extraction.

In addition to the acellular portion of the whole blood, DNA may also be recovered from the cellular fraction, enriched in the buffy coat portion, which can be obtained following centrifugation of a whole blood sample from the woman and removal of the plasma.

There are numerous known methods for extracting DNA from a biological sample including blood. General methods of DNA preparation can be followed using various commercially available reagents or kits, such as Qiagen's QIAamp Circulating Nucleic Acid Kit, QiaAmp DNA Mini Kit or

QiaAmp DNA Blood Mini Kit (Qiagen, Hilden, Germany), GenomicPrep™ Blood DNA Isolation Kit (Promega, Madison, Wis.), or GFX™ Genomic Blood DNA Purification Kit (Amersham, Piscataway, N.J.). Combinations of more than one of these methods may also be used.

Nucleic acid may be provided for conducting methods described herein without processing of the sample(s) containing the nucleic acid, in certain embodiments. In some embodiments, nucleic acid is provided for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. For example, fetal nucleic acid can be purified from a mixture comprising maternal and fetal nucleic acid. In certain examples, nucleosomes comprising small fragments of fetal nucleic acid can be purified from a mixture of larger nucleosome complexes comprising larger fragments of maternal nucleic acid.

In some embodiments, nucleic acids are fragmented or cleaved prior to, during or after a method described herein. Fragmented or cleaved nucleic acid may have a nominal, average or mean length of about 5 to about 10,000 base pairs, about 100 to about 1,000 base pairs, about 100 to about 500 base pairs, or about 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000 or 9000 base pairs. Fragments can be generated by a suitable method known in the art, and the average, mean or nominal length of nucleic acid fragments can be controlled by selecting an appropriate fragment-generating procedure.

In some embodiments, nucleic acid is fragmented or cleaved by a suitable method, non-limiting examples of which include physical methods (e.g., shearing, e.g., sonication, French press, heat,

UV irradiation, the like), enzymatic processes (e.g., enzymatic cleavage agents (e.g., a suitable nuclease, a suitable restriction enzyme, a suitable methylation sensitive restriction enzyme)), chemical methods (e.g., alkylation, DMS, piperidine, acid hydrolysis, base hydrolysis, heat, the like, or combinations thereof), the like or combinations thereof.

Nucleic acid also may be exposed to a process that modifies certain nucleotides in the nucleic acid before providing nucleic acid for a method described herein. A process that selectively modifies nucleic acid based upon the methylation state of nucleotides therein can be applied to nucleic acid, for example. In addition, conditions such as high temperature, ultraviolet radiation, x-radiation, can induce changes in the sequence of a nucleic acid molecule. Nucleic acid may be provided in any suitable form useful for conducting a suitable sequence analysis.

In some embodiments, a sample may first be enriched or relatively enriched for fetal nucleic acid by one or more methods. For example, the discrimination of fetal and maternal DNA can be performed using certain discriminating factors. Examples of these factors include, but are not limited to, single nucleotide differences between chromosome X and Y, chromosome Y-specific sequences, polymorphisms located elsewhere in the genome, size differences between fetal and maternal DNA, and differences in methylation pattern between maternal and fetal tissues. In certain applications, maternal nucleic acid is selectively removed (either partially, substantially, almost completely or completely) from the sample.

### Nucleic Acid

The terms “nucleic acid” and “nucleic acid molecule” may be used interchangeably throughout the disclosure. The terms refer to nucleic acids of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single-or double-stranded form, and unless otherwise limited, can encompass known analogs of natural nucleotides that can function in a similar manner as naturally occurring nucleotides. A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in certain instances. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame) and double-stranded polynucleotides. The term “gene” refers to the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons). Deoxyribonucleotides include deoxyadenosine, deoxycytidine, deoxyguanosine and deoxythymidine. For RNA, the base thymine is replaced with uracil. A template nucleic acid may be prepared using a nucleic acid obtained from a subject as a template.

Nucleic acids can include extracellular nucleic acid in certain embodiments. The term “extracellular nucleic acid” as used herein can refer to nucleic acid isolated from a source having substantially no cells and also is referred to as “cell-free” nucleic acid, “circulating cell-free nucleic acid” (e.g., CCF fragments) and/or “cell-free circulating nucleic acid”. Extracellular nucleic acid can be present in and obtained from blood (e.g., from the blood of a pregnant subject). Extracellular nucleic acid often includes no detectable cells and may contain cellular elements or cellular remnants. Non-limiting examples of acellular sources for extracellular nucleic acid are blood, blood plasma, blood serum, and urine. As used herein, the term “obtain cell-free circulating sample nucleic acid” includes obtaining a sample directly (e.g., collecting a sample, e.g., a test sample) or obtaining a sample from another who has collected a sample. Without being limited by theory, extracellular nucleic acid may be a product of cell apoptosis and cell breakdown, which provides basis for extracellular nucleic acid often having a series of lengths across a spectrum (e.g., a “ladder”).

Extracellular nucleic acid can include different nucleic acid species, and therefore is referred to herein as “heterogeneous” in certain embodiments. For example, blood serum or plasma from a person having cancer can include nucleic acid from cancer cells and nucleic acid from non-cancer cells. In another example, blood serum or plasma from a pregnant subject can include maternal nucleic acid and fetal nucleic acid. In some instances, fetal nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is fetal nucleic acid). In some embodiments, the majority of fetal nucleic acid in nucleic acid is of a length of about 500 base pairs or less (e.g., about 80, 85, 90, 91, 92, 93, 94, 95, 96, 97, 98, 99 or 100% of fetal nucleic acid is of a length of about 500 base pairs or less). In some embodiments, the majority of fetal nucleic acid in nucleic acid is of a length of about 250 base pairs or less (e.g., about 80, 85, 90, 91, 92, 93, 94, 95, 96, 97, 98, 99 or 100% of fetal nucleic acid is of a length of about 250 base pairs or less). In some embodiments, the majority of fetal nucleic acid in nucleic acid is of a length of about 200 base pairs or less (e.g., about 80, 85, 90, 91, 92, 93, 94, 95, 96, 97, 98, 99 or 100% of fetal nucleic acid is of a length of about 200 base pairs or less). In some embodiments, the majority of fetal nucleic acid in nucleic acid is of a length of about 150 base pairs or less (e.g., about 80, 85, 90, 91, 92, 93, 94, 95, 96, 97, 98, 99 or 100% of fetal nucleic acid is of a length of about 150 base pairs or less). In some embodiments, the majority of fetal nucleic acid in nucleic acid is of a length of about 100 base pairs or less (e.g., about 80, 85, 90, 91, 92, 93, 94, 95, 96, 97, 98, 99 or 100% of fetal nucleic acid is of a length of about 100 base pairs or less). In some embodiments, the majority of fetal nucleic acid in nucleic acid is of a length of about 50 base pairs or less (e.g., about 80, 85, 90, 91, 92, 93, 94, 95, 96, 97, 98, 99 or 100% of fetal nucleic acid is of a length of about 50 base pairs or less). In some embodiments, the majority of fetal nucleic acid in nucleic acid is of a length of about 25 base pairs or less (e.g., about 80, 85, 90, 91, 92, 93, 94, 95, 96, 97, 98, 99 or 100% of fetal nucleic acid is of a length of about 25 base pairs or less).

Nucleic acid may be single or double stranded. Single stranded DNA, for example, can be generated by denaturing double stranded DNA by heating or by treatment with alkali, for example. In certain embodiments, nucleic acid is in a D-loop structure, formed by strand invasion of a duplex DNA molecule by an oligonucleotide or a DNA-like molecule such as peptide nucleic acid (PNA). D loop formation can be facilitated by addition of E. Coli RecA protein and/or by alteration of salt concentration, for example, using methods known in the art.

### Nucleic Acid Library

In some embodiments, a nucleic acid library is a plurality of polynucleotide molecules (e.g., a sample of nucleic acids) that are prepared, assemble and/or modified for a specific process, non-limiting examples of which include immobilization on a solid phase (e.g., a solid support, e.g., a flow cell, a bead), enrichment, amplification, cloning, detection and/or for nucleic acid sequencing. In certain embodiments, a nucleic acid library is prepared prior to or during a sequencing process. A nucleic acid library (e.g., sequencing library) can be prepared by a suitable method as known in the art. A nucleic acid library can be prepared by a targeted or a non-targeted preparation process.

In some embodiments, a library of nucleic acids is modified to comprise a chemical moiety (e.g., a functional group) configured for immobilization of nucleic acids to a solid support. In some embodiments, a library of nucleic acids is modified to comprise a biomolecule (e.g., a functional group) and/or member of a binding pair configured for immobilization of the library to a solid support, non-limiting examples of which include thyroxin-binding globulin, steroid-binding proteins, antibodies, antigens, haptens, enzymes, lectins, nucleic acids, repressors, protein A, protein G, avidin, streptavidin, biotin, complement component C1q, nucleic acid-binding proteins, receptors, carbohydrates, oligonucleotides, polynucleotides, complementary nucleic acid sequences, the like and combinations thereof. Some examples of specific binding pairs include, without limitation: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; an oligonucleotide or polynucleotide and its corresponding complement; the like or combinations thereof.

In some embodiments, a library of nucleic acids is modified to comprise one or more polynucleotides of known composition, non-limiting examples of which include an identifier (e.g., a tag, an indexing tag), a capture sequence, a label, an adapter, a restriction enzyme site, a promoter, an enhancer, an origin of replication, a stem loop, a complimentary sequence (e.g., a primer binding site, an annealing site), a suitable integration site (e.g., a transposon, a viral integration site), a modified nucleotide, the like or combinations thereof. Polynucleotides of known sequence can be added at a suitable position, for example on the 5′ end, 3′ end or within a nucleic acid sequence. Polynucleotides of known sequence can be the same or different sequences. In some embodiments, a polynucleotide of known sequence is configured to hybridize to one or more oligonucleotides immobilized on a surface (e.g., a surface in flow cell). For example, a nucleic acid molecule comprising a 5′ known sequence may hybridize to a first plurality of oligonucleotides while the 3′ known sequence may hybridize to a second plurality of oligonucleotides. In some embodiments, a library of nucleic acid can comprise chromosome-specific tags, capture sequences, labels and/or adaptors. In some embodiments, a library of nucleic acids comprises one or more detectable labels. In some embodiments, one or more detectable labels may be incorporated into a nucleic acid library at a 5′ end, at a 3′ end, and/or at any nucleotide position within a nucleic acid in the library. In some embodiments, a library of nucleic acids comprises hybridized oligonucleotides. In certain embodiments, hybridized oligonucleotides are labeled probes. In some embodiments, a library of nucleic acids comprises hybridized oligonucleotide probes prior to immobilization on a solid phase.

In some embodiments, a polynucleotide of known sequence comprises a universal sequence. A universal sequence is a specific nucleotide acid sequence that is integrated into two or more nucleic acid molecules or two or more subsets of nucleic acid molecules where the universal sequence is the same for all molecules or subsets of molecules that it is integrated into. A universal sequence is often designed to hybridize to and/or amplify a plurality of different sequences using a single universal primer that is complementary to a universal sequence. In some embodiments, two (e.g., a pair) or more universal sequences and/or universal primers are used. A universal primer often comprises a universal sequence. In some embodiments, adapters (e.g., universal adapters) comprise universal sequences. In some embodiments, one or more universal sequences are used to capture, identify and/or detect multiple species or subsets of nucleic acids.

In certain embodiments of preparing a nucleic acid library, (e.g., in certain sequencing by synthesis procedures), nucleic acids are size selected and/or fragmented into lengths of several hundred base pairs, or less (e.g., in preparation for library generation). In some embodiments, library preparation is performed without fragmentation (e.g., when using ccfDNA). For example, certain methods described herein identify native end motifs in ccfDNA fragments. Accordingly, libraries for such methods are generated using the native fragments from a sample and are not subjected to a fragmentation process.

In certain embodiments, a ligation-based library preparation method is used (e.g., ILLUMINA TRUSEQ, Illumina, San Diego CA). Ligation-based library preparation methods often make use of an adaptor (e.g., a methylated adaptor) design which can incorporate an index sequence at the initial ligation step and often can be used to prepare samples for single-read sequencing, paired-end sequencing, and/or multiplexed sequencing. For example, nucleic acids (e.g., fragmented nucleic acids or ccfDNA) may be end repaired by a fill-in reaction, an exonuclease reaction, or a combination thereof. In certain configurations, 5′ fragment ends are end repaired by a fill-in reaction and 3′ fragment ends are end repaired by an exonuclease reaction (e.g., a 3′ to 5′ single-stranded exonuclease). Specifically, fragment ends with 5′ overhangs are end repaired by a fill-in reaction and fragment ends with 3′ overhangs are end repaired by an exonuclease reaction. In such configurations, end motif sequences are retained on 5′ fragment ends. In some embodiments, the resulting blunt-end repaired nucleic acid can then be extended by a single nucleotide, which is complementary to a single nucleotide overhang on the 3′ end of an adapter/primer. Any nucleotide can be used for the extension/overhang nucleotides. In some embodiments, nucleic acid library preparation comprises ligating an adapter oligonucleotide. Adapter oligonucleotides are often complementary to flow-cell anchors, and may be utilized to immobilize a nucleic acid library to a solid support, such as the inside surface of a flow cell, for example. In some embodiments, an adapter oligonucleotide comprises an identifier, one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing).

An identifier can be a suitable detectable label incorporated into or attached to a nucleic acid (e.g., a polynucleotide) that allows detection and/or identification of nucleic acids that comprise the identifier. In some embodiments, an identifier is incorporated into or attached to a nucleic acid during a sequencing method (e.g., by a polymerase). Non-limiting examples of identifiers include nucleic acid tags, nucleic acid indexes or barcodes, a radiolabel (e.g., an isotope), metallic label, a fluorescent label, a chemiluminescent label, a phosphorescent label, a fluorophore quencher, a dye, a protein (e.g., an enzyme, an antibody or part thereof, a linker, a member of a binding pair), the like or combinations thereof. In some embodiments, an identifier (e.g., a nucleic acid index or barcode) is a unique, known and/or identifiable sequence of nucleotides or nucleotide analogues. In some embodiments, identifiers are six or more contiguous nucleotides. A multitude of fluorophores are available with a variety of different excitation and emission spectra. Any suitable type and/or number of fluorophores can be used as an identifier. In some embodiments, 1 or more, 2 or more, 3 or more, 4 or more, 5 or more, 6 or more, 7 or more, 8 or more, 9 or more, 10 or more, 20 or more, 30 or more or 50 or more different identifiers are utilized in a method described herein (e.g., a nucleic acid detection and/or sequencing method). In some embodiments, one or two types of identifiers (e.g., fluorescent labels) are linked to each nucleic acid in a library. Detection and/or quantification of an identifier can be performed by a suitable method, apparatus or machine, non-limiting examples of which include flow cytometry, quantitative polymerase chain reaction (qPCR), gel electrophoresis, a luminometer, a fluorometer, a spectrophotometer, a suitable gene-chip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof.

In some embodiments, a nucleic acid library or parts thereof are amplified (e.g., amplified by a PCR-based method). In some embodiments, a sequencing method comprises amplification of a nucleic acid library. A nucleic acid library can be amplified prior to or after immobilization on a solid support (e.g., a solid support in a flow cell). Nucleic acid amplification includes the process of amplifying or increasing the numbers of a nucleic acid template and/or of a complement thereof that are present (e.g., in a nucleic acid library), by producing one or more copies of the template and/or its complement. Amplification can be carried out by a suitable method. A nucleic acid library can be amplified by a thermocycling method or by an isothermal amplification method. In some embodiments, a rolling circle amplification method is used. In some embodiments, amplification takes place on a solid support (e.g., within a flow cell) where a nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments, of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments, solid phase amplification comprises a nucleic acid amplification reaction comprising only one species of oligonucleotide primer immobilized to a surface. In certain embodiments solid phase amplification comprises a plurality of different immobilized oligonucleotide primer species. In some embodiments, solid phase amplification may comprise a nucleic acid amplification reaction comprising one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WILDFIRE amplification, the like or combinations thereof.

### Sequencing

In some embodiments, nucleic acids (e.g., nucleic acid fragments, sample nucleic acid, test sample nucleic acid, cell-free nucleic acid, circulating cell-free nucleic acid) are sequenced. In some embodiments, a full or substantially full sequence is obtained and sometimes a partial sequence is obtained. In some embodiments, a nucleic acid is not sequenced, and the sequence of a nucleic acid is not determined by a sequencing method, when performing a method described herein. In some embodiments, fragment length is determined using a sequencing method. In some embodiments, fragment length is determined without use of a sequencing method. In certain embodiments a non-targeted sequencing approach is used where most or all nucleic acids in a sample are sequenced, amplified and/or captured randomly. Certain aspects of sequencing and analysis processes are described hereafter.

In some embodiments, fragment length is determined using a sequencing method. In some embodiments, fragment length is determined using a paired-end sequencing platform. Such platforms involve sequencing of both ends of a nucleic acid fragment. Generally, the sequences corresponding to both ends of the fragment can be mapped to a reference genome (e.g., a reference human genome). In certain embodiments, both ends are sequenced at a read length that is sufficient to map, individually for each fragment end, to a reference genome. Examples of paired-end sequence read lengths are described below. In certain embodiments, all or a portion of the sequence reads can be mapped to a reference genome without mismatch. In some embodiments, each read is mapped independently. In some embodiments, information from both sequence reads (i.e., from each end) is factored in the mapping process. The length of a fragment can be determined, for example, by calculating the difference between genomic coordinates assigned to each mapped paired-end read.

In some embodiments, fragment length can be determined using a sequencing process whereby a complete, or substantially complete, nucleotide sequence is obtained for the fragment. Such sequencing processes include platforms that generate relatively long read lengths (e.g., Roche 454, Ion Torrent, single molecule (Pacific Biosciences), real-time SMRT technology, and the like).

In some embodiments, some or all nucleic acids in a sample are enriched and/or amplified (e.g., non-specifically, e.g., by a PCR based method) prior to or during sequencing. In certain embodiments, specific nucleic acid portions or subsets in a sample are enriched and/or amplified prior to or during sequencing. In some embodiments, a portion or subset of a pre-selected pool of nucleic acids is sequenced randomly. In some embodiments, nucleic acids in a sample are not enriched and/or amplified prior to or during sequencing.

As used herein, “reads” (i.e., “a read”, “a sequence read”) are short nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). A sequence read can include a fragment end sequence associated with an end of a nucleic acid fragment. The fragment end sequence can correspond to the outermost N bases of a nucleic acid fragment, e.g., 2-30 bases at the end of the nucleic acid fragment. If a sequence read corresponds to an entire nucleic acid fragment, then the sequence read can include two fragment end sequences. When paired-end sequencing generates two sequence reads that correspond to the ends of the fragments, each sequence read can include one fragment end sequence.

The length of a sequence read is often associated with the particular sequencing technology. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments sequence reads are of a mean, median, average or absolute length about 1000 bp or more.

In some embodiments, the nominal, average, mean or absolute length of single-end reads sometimes is about 1 nucleotide to about 500 contiguous nucleotides, about 15 contiguous nucleotides to about 50 contiguous nucleotides, about 30 contiguous nucleotides to about 40 contiguous nucleotides, and sometimes about 35 contiguous nucleotides or about 36 contiguous nucleotides. In certain embodiments the nominal, average, mean or absolute length of single-end reads is about 20 to about 30 bases, or about 24 to about 28 bases in length. In certain embodiments the nominal, average, mean or absolute length of single-end reads is about 1, 2, 3, 4, 5, 6, 7, 8, 9, 10, 11, 12, 13,14, 15, 16, 17, 18, 19, 21, 22, 23, 24, 25, 26, 27, 28, 29, 30, 31, 32, 33, 34, 35, 36, 37, 38, 39, 40, 41, 42, 43, 44, 45, 46, 47, 48 or 49 bases in length.

In certain embodiments, the nominal, average, mean or absolute length of paired-end reads sometimes is about 10 contiguous nucleotides to about 25 contiguous nucleotides (e.g., about 10, 11, 12, 13, 14, 15, 16, 17, 18, 19, 20, 21, 22, 23, 24 or 25 nucleotides in length), about 15 contiguous nucleotides to about 20 contiguous nucleotides, and sometimes is about 17 contiguous nucleotides, about 18 contiguous nucleotides, about 20 contiguous nucleotides, about 25 contiguous nucleotides, about 36 contiguous nucleotides or about 45 contiguous nucleotides.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid. Sequence reads obtained from the blood, plasma, or serum of a pregnant subject can be reads from a mixture of fetal and maternal nucleic acid. A mixture of relatively short reads can be transformed by processes described herein into a representation of a genomic nucleic acid present in the pregnant subject and/or in the fetus. A mixture of relatively short reads can be transformed into a representation of a copy number variation (e.g., a maternal and/or fetal copy number variation), genetic variation or an aneuploidy, for example. Reads of a mixture of maternal and fetal nucleic acid can be transformed into a representation of a composite chromosome or a segment thereof comprising features of one or both maternal and fetal chromosomes. In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

In some embodiments, a representative fraction of a genome is sequenced and is sometimes referred to as “coverage” or “fold coverage.” For example, a 1-fold coverage indicates that roughly 100% of the nucleotide sequences of the genome are represented by reads. In some instances, fold coverage is referred to as (and is directly proportional to) “sequencing depth.” In some embodiments, “fold coverage” is a relative term referring to a prior sequencing run as a reference. For example, a second sequencing run may have 2-fold less coverage than a first sequencing run. In some embodiments, a genome is sequenced with redundancy, where a given region of the genome can be covered by two or more reads or overlapping reads (e.g., a “fold coverage” greater than 1, e.g., a 2-fold coverage). In some embodiments, a genome (e.g., a whole genome) is sequenced with about 0.01-fold to about 100-fold coverage, about 0.1-fold to 20-fold coverage, or about 0.1-fold to about 1-fold coverage (e.g., about 0.015-, 0.02-, 0.03-, 0.04-, 0.05-, 0.06-, 0.07-, 0.08-, 0.09-, 0.1-, 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold or greater coverage). In some embodiments, specific parts of a genome (e.g., genomic parts from targeted methods) are sequenced and fold coverage values generally refer to the fraction of the specific genomic parts sequenced (i.e., fold coverage values do not refer to the whole genome). In some instances, specific genomic parts are sequenced at 1000-fold coverage or more. For example, specific genomic parts may be sequenced at 2000-fold, 5,000-fold, 10,000-fold, 20,000-fold, 30,000-fold, 40,000-fold or 50,000-fold coverage. In some embodiments, sequencing is at about 1,000-fold to about 100,000-fold coverage. In some embodiments, sequencing is at about 10,000-fold to about 70,000-fold coverage. In some embodiments, sequencing is at about 20,000-fold to about 60,000-fold coverage. In some embodiments, sequencing is at about 30,000-fold to about 50,000-fold coverage.

In certain embodiments, a subset of nucleic acid fragments is selected prior to sequencing. In certain embodiments, hybridization-based techniques (e.g., using oligonucleotide arrays) can be used to first select for nucleic acid sequences from certain chromosomes. In some embodiments, nucleic acid can be fractionated by size (e.g., by gel electrophoresis, size exclusion chromatography or by microfluidics-based approach) and in certain instances, fetal nucleic acid can be enriched by selecting for nucleic acid having a lower molecular weight (e.g., less than 300 base pairs, less than 200 base pairs, less than 150 base pairs, less than 100 base pairs). In some embodiments, fetal nucleic acid can be enriched by suppressing maternal background nucleic acid, such as by the addition of formaldehyde. In some embodiments, a portion or subset of a pre-selected set of nucleic acid fragments is sequenced randomly. In some embodiments, the nucleic acid is amplified prior to sequencing. In some embodiments, a portion or subset of the nucleic acid is amplified prior to sequencing.

In some embodiments, one nucleic acid sample from one individual is sequenced. In certain embodiments, nucleic acids from each of two or more samples are sequenced, where samples are from one individual or from different individuals. In certain embodiments, nucleic acid samples from two or more biological samples are pooled, where each biological sample is from one individual or two or more individuals and the pool is sequenced. In the latter embodiments, a nucleic acid sample from each biological sample often is identified by one or more unique identifiers or identification tags.

In some embodiments, a sequencing method utilizes identifiers that allow multiplexing of sequence reactions in a sequencing process. The greater the number of unique identifiers, the greater the number of samples and/or chromosomes for detection, for example, that can be multiplexed in a sequencing process. A sequencing process can be performed using any suitable number of unique identifiers (e.g., 4, 8, 12, 24, 48, 96, or more).

A sequencing process sometimes makes use of a solid phase, and sometimes the solid phase comprises a flow cell on which nucleic acid from a library can be attached and reagents can be flowed and contacted with the attached nucleic acid. A flow cell sometimes includes flow cell lanes, and use of identifiers can facilitate analyzing a number of samples in each lane. A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs. In some embodiments, the number of samples analyzed in a given flow cell lane are dependent on the number of unique identifiers utilized during library preparation and/or probe design. Multiplexing using 12 identifiers, for example, allows simultaneous analysis of 96 samples (e.g., equal to the number of wells in a 96 well microwell plate) in an 8-lane flow cell. Similarly, multiplexing using 48 identifiers, for example, allows simultaneous analysis of 384 samples (e.g., equal to the number of wells in a 384 well microwell plate) in an 8-lane flow cell. Non-limiting examples of commercially available multiplex sequencing kits include Illumina's multiplexing sample preparation oligonucleotide kit and multiplexing sequencing primers and PhiX control kit (e.g., Illumina's catalog numbers PE-400-1001 and PE-400-1002, respectively).

Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In some embodiments, MPS sequencing methods utilize a targeted approach, where specific chromosomes, genes or regions of interest are sequences. In certain embodiments a non-targeted MPS approach is used where most or all nucleic acids in a sample are sequenced, amplified and/or captured randomly.

A suitable MPS method, system or technology platform for conducting methods described herein can be used to obtain nucleic acid sequencing reads. Non-limiting examples of MPS platforms include Illumina/Solex/HiSeq (e.g., Illumina's Genome Analyzer; Genome Analyzer II; HISEQ 2000; HISEQ), SOLID, Roche/454, PACBIO and/or SMRT, Helicos True Single Molecule Sequencing, Ion Torrent and Ion semiconductor-based sequencing (e.g., as developed by Life Technologies), WildFire, 5500, 5500xl W and/or 5500xl W Genetic Analyzer based technologies (e.g., as developed and sold by Life Technologies, US patent publication No. US20130012399); Polony sequencing, Pyrosequencing, Massively Parallel Signature Sequencing (MPSS), RNA polymerase (RNAP) sequencing, LaserGen systems and methods, Nanopore-based platforms, chemical-sensitive field effect transistor (CHEMFET) array, electron microscopy-based sequencing (e.g., as developed by ZS Genetics, Halcyon Molecular), and nanoball sequencing.

MPS sequencing sometimes makes use of sequencing by synthesis and certain imaging processes. A nucleic acid sequencing technology that may be used in a method described herein is sequencing-by-synthesis and reversible terminator-based sequencing (e.g., Illumina's Genome Analyzer; Genome Analyzer II; HISEQ 2000; HISEQ 2500 (Illumina, San Diego CA)). With this technology, thousands to millions of nucleic acid (e.g. DNA) fragments can be sequenced in parallel. In one example of this type of sequencing technology, a flow cell is used which contains an optically transparent slide with 8 individual lanes on the surfaces of which are bound oligonucleotide anchors (e.g., adaptor primers). A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs.

Sequencing by synthesis, in some embodiments, comprises iteratively adding (e.g., by covalent addition) a nucleotide to a primer or preexisting nucleic acid strand in a template directed manner. Each iterative addition of a nucleotide is detected and the process is repeated multiple times until a sequence of a nucleic acid strand is obtained. The length of a sequence obtained depends, in part, on the number of addition and detection steps that are performed. In some embodiments, of sequencing by synthesis, one, two, three or more nucleotides of the same type (e.g., A, G, C or T) are added and detected in a round of nucleotide addition. Nucleotides can be added by any suitable method (e.g., enzymatically or chemically). For example, in some embodiments, a polymerase or a ligase adds a nucleotide to a primer or to a preexisting nucleic acid strand in a template directed manner. In some embodiments, of sequencing by synthesis, different types of nucleotides, nucleotide analogues and/or identifiers are used. In some embodiments, reversible terminators and/or removable (e.g., cleavable) identifiers are used. In some embodiments, fluorescent labeled nucleotides and/or nucleotide analogues are used. In certain embodiments sequencing by synthesis comprises a cleavage (e.g., cleavage and removal of an identifier) and/or a washing step. In some embodiments, the addition of one or more nucleotides is detected by a suitable method described herein or known in the art, non-limiting examples of which include any suitable imaging apparatus or machine, a suitable camera, a digital camera, a CCD (Charge Couple Device) based imaging apparatus (e.g., a CCD camera), a CMOS (Complementary Metal Oxide Silicon) based imaging apparatus (e.g., a CMOS camera), a photo diode (e.g., a photomultiplier tube), electron microscopy, a field-effect transistor (e.g., a DNA field-effect transistor), an ISFET ion sensor (e.g., a CHEMFET sensor), the like or combinations thereof. Other sequencing methods that may be used to conduct methods herein include digital PCR and sequencing by hybridization.

Other sequencing methods that may be used to conduct methods herein include digital PCR and sequencing by hybridization. Digital polymerase chain reaction (digital PCR or dPCR) can be used to directly identify and quantify nucleic acids in a sample. Digital PCR can be performed in an emulsion, in some embodiments. For example, individual nucleic acids are separated, e.g., in a microfluidic chamber device, and each nucleic acid is individually amplified by PCR. Nucleic acids can be separated such that there is no more than one nucleic acid per well. In some embodiments, different probes can be used to distinguish various alleles (e.g., fetal alleles and maternal alleles). Alleles can be enumerated to determine copy number.

In certain embodiments, sequencing by hybridization can be used. The method involves contacting a plurality of polynucleotide sequences with a plurality of polynucleotide probes, where each of the plurality of polynucleotide probes can be optionally tethered to a substrate. The substrate can be a flat surface with an array of known nucleotide sequences, in some embodiments. The pattern of hybridization to the array can be used to determine the polynucleotide sequences present in the sample. In some embodiments, each probe is tethered to a bead, e.g., a magnetic bead or the like. Hybridization to the beads can be identified and used to identify the plurality of polynucleotide sequences within the sample.

In some embodiments, nanopore sequencing can be used in a method described herein. Nanopore sequencing is a single-molecule sequencing technology whereby a single nucleic acid molecule (e.g., DNA) is sequenced directly as it passes through a nanopore.

In some embodiments, a targeted enrichment, amplification and/or sequencing approach is used. A targeted approach often isolates, selects and/or enriches a subset of nucleic acids in a sample for further processing by use of sequence-specific oligonucleotides. In some embodiments, a library of sequence-specific oligonucleotides is utilized to target (e.g., hybridize to) one or more sets of nucleic acids in a sample. Sequence-specific oligonucleotides and/or primers are often selective for particular sequences (e.g., unique nucleic acid sequences) present in one or more chromosomes, genes, exons, introns, and/or regulatory regions of interest. Any suitable method or combination of methods can be used for enrichment, amplification and/or sequencing of one or more subsets of targeted nucleic acids. In some embodiments, targeted sequences are isolated and/or enriched by capture to a solid phase (e.g., a flow cell, a bead) using one or more sequence-specific anchors. In some embodiments, targeted sequences are enriched and/or amplified by a polymerase-based method (e.g., a PCR-based method, by any suitable polymerase-based extension) using sequence-specific primers and/or primer sets. Sequence specific anchors often can be used as sequence-specific primers.

### Mapping Reads

In some embodiments, sequence reads are mapped to a genome (e.g., a reference genome) or part thereof. Any suitable mapping method (e.g., process, algorithm, program, software, module, the like or combination thereof) can be used. Certain aspects of mapping processes are described hereafter.

Mapping nucleotide sequence reads (i.e., sequence information from a fragment whose physical genomic position is unknown) can be performed in a number of ways, and often comprises alignment of the obtained sequence reads with a matching sequence in a reference genome. In such alignments, sequence reads generally are aligned to a reference sequence and those that align are designated as being “mapped”, “a mapped sequence read” or “a mapped read”. In certain embodiments, a mapped sequence read is referred to as a “hit” or “count”. In some embodiments, mapped sequence reads are grouped together according to various parameters, as described herein. In some embodiments, mapped sequence reads are assigned to particular chromosomes or portions thereof.

As used herein, the terms “aligned”, “alignment”, or “aligning” refer to two or more nucleic acid sequences that can be identified as a match (e.g., 100% identity) or partial match. Alignments can be done manually or by a computer (e.g., a software, program, module, or algorithm), non-limiting examples of which include the Efficient Local Alignment of Nucleotide Data (ELAND) computer program distributed as part of the Illumina Genomics Analysis pipeline. Alignment of a sequence read can be a 100% sequence match. In some cases, an alignment is less than a 100% sequence match (i.e., non-perfect match, partial match, partial alignment). In some embodiments, an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4 or 5 mismatches. Two or more sequences can be aligned using either strand. In certain embodiments a nucleic acid sequence is aligned with the reverse complement of another nucleic acid sequence.

Various computational methods can be used to map each sequence read to a reference genome, or portion thereof. Non-limiting examples of computer algorithms that can be used to align sequences include, without limitation, BLAST, BLITZ, FASTA, BOWTIE 1, BOWTIE 2, ELAND, MAQ, PROBEMATCH, SOAP or SEQMAP, or variations thereof or combinations thereof. In some embodiments, sequence reads can be aligned with sequences in a reference genome. In some embodiments, the sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search the identified sequences against a sequence database. Search hits can then be used to sort the identified sequences into appropriate chromosomes or genomic portions, for example.

In some embodiments, a read may uniquely or non-uniquely map to a reference genome, or portion thereof. A read is considered as “uniquely mapped” if it aligns with a single sequence in the reference genome. A read is considered as “non-uniquely mapped” if it aligns with two or more sequences in the reference genome. In some embodiments, non-uniquely mapped reads are eliminated from further analysis (e.g., fragment length measurements, sequence motif quantifications). A certain, small degree of mismatch (0-1) may be allowed to account for single nucleotide polymorphisms that may exist between the reference genome and the reads from individual samples being mapped, in certain embodiments. In some embodiments, no degree of mismatch is allowed for a read mapped to a reference sequence.

As used herein, the term “reference genome” can refer to any particular known, sequenced or characterized genome, whether partial or complete, of any organism or virus which may be used to reference identified sequences from a subject. For example, a reference genome used for human subjects as well as many other organisms can be found at the National Center for Biotechnology Information at www.ncbi.nlm.nih.gov. A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. In some embodiments, a reference genome comprises sequences assigned to chromosomes.

In certain embodiments, where a sample nucleic acid is from a pregnant subject, a reference sequence sometimes is not from the fetus, the mother of the fetus or the father of the fetus, and is referred to herein as an “external reference.” A maternal reference may be prepared and used in some embodiments. When a reference from the pregnant female is prepared (“maternal reference sequence”) based on an external reference, reads from DNA of the pregnant female that contains substantially no fetal DNA often are mapped to the external reference sequence and assembled. In certain embodiments the external reference is from DNA of an individual having substantially the same ethnicity as the pregnant female. A maternal reference sequence may not completely cover the maternal genomic DNA (e.g., it may cover about 50%, 60%, 70%, 80%, 90% or more of the maternal genomic DNA), and the maternal reference may not perfectly match the maternal genomic DNA sequence (e.g., the maternal reference sequence may include multiple mismatches).

In certain embodiments, mappability is assessed for a reference genome or genomic region (e.g., portion, genomic portion, portion). Mappability is the ability to unambiguously align a nucleotide sequence read to a reference genome or a portion of a reference genome, typically up to a specified number of mismatches, including, for example, 0, 1, 2 or more mismatches. For a given genomic region, the expected mappability can be estimated using a sliding-window approach of a preset read length and averaging the resulting read-level mappability values. Genomic regions comprising stretches of unique nucleotide sequence sometimes have a high mappability value.

### Sequence Read Quantifications

Sequence reads that are mapped or partitioned based on a selected feature or variable can be quantified to determine the number of reads that are mapped to one or more portions, sections, partitions, loci (e.g., portions, sections, partitions, loci of a reference genome), in some embodiments. In certain embodiments, the quantity of sequence reads that are mapped to a portion are termed counts (e.g., a count). Often a count is associated with a portion. In certain embodiments counts for two or more portions (e.g., a set of portions) are mathematically manipulated (e.g., averaged, added, normalized, the like or a combination thereof). In some embodiments a count is determined from some or all of the sequence reads mapped to (i.e., associated with) a portion. In certain embodiments, a count is determined from a pre-defined subset of mapped sequence reads. Pre-defined subsets of mapped sequence reads can be defined or selected utilizing any suitable feature or variable. In some embodiments, pre-defined subsets of mapped sequence reads can include from 1 to n sequence reads, where n represents a number equal to the sum of all sequence reads generated from a test subject or reference subject sample.

In certain embodiments, a sequence read quantification or count is derived from sequence reads that are processed or manipulated by a suitable method, operation or mathematical process known in the art. A quantification or count can be determined by a suitable method, operation or mathematical process. In certain embodiments a quantification or count is derived from sequence reads associated with a portion where some or all of the sequence reads are weighted, removed, filtered, normalized, adjusted, averaged, derived as a mean, added, or subtracted or processed by a combination thereof. In some embodiments, a quantification or count is derived from raw sequence reads and or filtered sequence reads. In certain embodiments, a quantification or count value is determined by a mathematical process. In certain embodiments, a quantification or count value is an average, mean or sum of sequence reads mapped to a portion. In some embodiments, a quantification or count is a mean number of counts. In some embodiments, a quantification or count is associated with an uncertainty value.

In some embodiments, sequence read quantifications or counts can be manipulated or transformed (e.g., normalized, combined, added, filtered, selected, averaged, derived as a mean, the like, or a combination thereof). In some embodiments, quantifications or counts can be transformed to produce normalized counts. Quantifications or counts can be processed (e.g., normalized) by a method known in the art and/or as described herein (e.g., portion-wise normalization, normalization by GC content, linear and nonlinear least squares regression, GC LOESS, LOWESS, RM, GCRM, cQn and/or combinations thereof).

Sequence read quantifications or counts may be obtained from a nucleic acid sample from a pregnant subject bearing a fetus. Quantifications or counts of nucleic acid sequence reads mapped to one or more portions often are quantifications or counts representative of both the fetus and the mother of the fetus (e.g., a pregnant subject). In certain embodiments, some of the quantifications or counts mapped to a portion are from a fetal genome and some of the counts mapped to the same portion are from a maternal genome.

### Fragment Length Measurement

In some embodiments, length is measured for one or more nucleic acid fragments (e.g., one or more cell-free nucleic acid fragments; one or more circulating cell-free nucleic acid fragments). In some embodiments, a sequence-based fragment length measurement is used. For example, nucleic acid fragment length may be measured using a paired-end sequencing platform. Such platforms involve sequencing of both ends of a nucleic acid fragment. Generally, the sequences corresponding to both ends of the nucleic acid fragment can be mapped to a reference genome (e.g., a reference human genome). In certain embodiments, both ends are sequenced at a read length that is sufficient to map, individually for each fragment end, to a reference genome. In certain embodiments, all or a portion of the sequence reads can be mapped to a reference genome without mismatch. In some embodiments, each read is mapped independently. In some embodiments, information from both sequence reads (i.e., from each end of a nucleic acid fragment) is factored in the mapping process. The length of a nucleic acid fragment can be measured, for example, by calculating the difference between genomic coordinates assigned to each mapped paired-end read. In other words, the length of a nucleic acid fragment can be measured (e.g., deduced or inferred) by mapping two or more reads derived from the nucleic acid fragment (e.g., a paired-end read) to a reference genome. For paired-end reads derived from a nucleic acid fragment, for example, reads can be mapped to a reference genome, the length of the genomic sequence between the mapped reads can be determined, and the total of the two read lengths and the length of the genomic sequence between the reads is equal to the length of the nucleic acid fragment. In some embodiments, the length of a nucleic acid fragment is measured directly from the length of a read derived from the fragment (e.g., single-end read).

In certain applications of a method described herein, nucleic acid fragment length can be measured using any method in the art suitable for determining nucleic acid fragment length, such as, for example, a mass sensitive process (e.g., mass spectrometry (e.g., matrix-assisted laser desorption ionization (MALDI) mass spectrometry and electrospray (ES) mass spectrometry), electrophoresis (e.g., capillary electrophoresis), microscopy (scanning tunneling microscopy, atomic force microscopy), and measuring length using a nanopore. In some applications, fragment length may be determined by measuring the length of a probe that hybridizes to the fragment.

### Genomic Intervals and Segments

In some embodiments, data obtained from mapped sequence reads (e.g., fragment lengths, sequence motifs) are grouped together according to parts of a genome (e.g., parts of a reference genome). A part of a genome may also be referred to herein as a chromosome, genomic segment, genomic interval, genomic portion, genomic section, bin, region, partition, portion of a reference genome, or portion of a chromosome “In some embodiments, a part of a genome is an entire chromosome, a segment of a chromosome, a segment of a reference genome, a segment spanning multiple chromosomes, multiple chromosome segments, and/or combinations thereof. In some embodiments, a part of a genome is predefined based on specific parameters (e.g., a predefined length). In some embodiments, a part of a genome is arbitrarily or non-arbitrarily defined based on partitioning of a genome (e.g., partitioned by size, GC content, contiguous regions, contiguous regions of an arbitrarily defined size, and the like).

In some embodiments, a part of a genome (e.g., genomic interval, genomic segment) is delineated based on one or more parameters which include, for example, length or a particular feature or features of the sequence. Parts of a genome (e.g., genomic intervals, genomic segments) can be selected, filtered and/or removed from consideration using any suitable criteria know in the art or described herein. In some embodiments, a part of a genome (e.g., genomic interval, genomic segment) is based on a particular length of genomic sequence. Parts of a genome (e.g., genomic intervals, genomic segments) can be approximately the same length or parts of a genome (e.g., genomic intervals, genomic segments) can be different lengths. In some embodiments, parts of a genome (e.g., genomic intervals, genomic segments) are of about equal length.

In some embodiments, a part of a genome (e.g., genomic interval, genomic segment) can be a particular chromosome, parts of a chromosome of interest, such as, for example, a chromosome where a genetic variation is assessed (e.g., an aneuploidy of chromosomes 13, 18 and/or 21 or a sex chromosome). Parts of a genome (e.g., genomic intervals, genomic segments) can be genes, gene fragments, regulatory sequences, introns, exons, and the like.

In some embodiments, a genome (e.g., human genome, reference genome) is partitioned (e.g., into genomic intervals, genomic segments) based on information content of particular regions. In some embodiments, partitioning a genome may eliminate similar regions (e.g., identical or homologous regions or sequences) across the genome and only keep unique regions. Regions removed during partitioning may be within a single chromosome or may span multiple chromosomes. In some embodiments, a partitioned genome is trimmed down and optimized for faster alignment, often allowing for focus on uniquely identifiable sequences. In some embodiments, partitioning of a genome may be based on other criteria, such as, for example, speed/convenience while aligning tags, GC content (e.g., high or low GC content), uniformity of GC content, other measures of sequence content (e.g. fraction of individual nucleotides, fraction of pyrimidines or purines, fraction of natural vs. non-natural nucleic acids, fraction of methylated nucleotides, and CpG content), methylation state, duplex melting temperature, amenability to sequencing or PCR, uncertainty value assigned to individual portions of a reference genome, and/or a targeted search for particular features.

In some embodiments, fragment lengths are measured for a plurality of genomic intervals. Specifically, fragment lengths obtained by paired end reads mapping to chosen genomic intervals are measured. Fragment length measurements obtained by paired end reads mapping to genomic intervals may be obtained for each chosen interval. Fragment length ratios may be determined for each chosen interval, as described below. In some embodiments, a genomic interval is about 10 kilobases (kb) in length to about 500 kb in length, about 10 kb in length to about 200 kb in length, or about 50 kb in length to about 150 kb in length. For example, a genomic interval may be about 50 kb, 60 kb, 70 kb, 80 kb, 90 kb, 100 kb, 110 kb, 120 kb, 130 kb, 140 kb, or 150 kb in length. In some embodiments, a genomic interval is about 100 kb in length. A genomic interval is not limited to contiguous runs of sequence. Thus, genomic intervals can be made up of contiguous and/or non-contiguous sequences. A genomic interval is not limited to a single chromosome. In some embodiments, a genomic interval includes all or part of one chromosome or all or part of two or more chromosomes. In some embodiments, genomic intervals may span one, two, or more entire chromosomes. In addition, genomic intervals may span jointed or disjointed regions of multiple chromosomes.

In some embodiments, a fragment length profile is generated for a genomic segment. In some embodiments, one or more fragment length profiles are generated for one or more genomic segments. A segment can refer to a segment of a genome and/or a segment of a chromosome. In some embodiments, a segment is an entire chromosome. A segment of a genome or chromosome often contains a larger number of nucleotides than a genomic interval. Generally, a genomic segment is made up of a plurality of smaller genomic intervals. In some embodiments, a genomic segment is about 1 megabase (Mb) in length to about 10 megabases (Mb) in length. For example, a genomic segment may be about 1 Mb, 2 Mb, 3 Mb, 4 Mb, 5 Mb, 6 Mb, 7 Mb, 8 Mb, 9 Mb, or 10 Mb in length. In some embodiments, a genomic segment is about 5 Mb in length.

### Profiles

In some embodiments, a method herein comprises generating one or more profiles (e.g., profile plot) from various aspects of a data set or derivation thereof (e.g., product of one or more mathematical and/or statistical data processing steps known in the art and/or described herein). In some embodiments, a method herein comprises generating one or more fragment length profiles. A fragment length profile may be generated for a genomic segment as described herein. A fragment length profile may be generated for a genomic segment partitioned into smaller genomic intervals as described herein. A fragment length value may be assigned to each genomic interval for a profile. A fragment length value may be a raw fragment length or a derivative thereof, e.g., a fragment length ratio, a median fragment length, a mean fragment length, an average fragment length, a normalized fragment length, and the like.

In some embodiments, a fragment length profile is generated according to fragment length ratios (i.e., fragment length rations determined for a plurality of genomic intervals). In some embodiments, a fragment length profile is generated according to ratios of X to Y for a plurality of genomic intervals, where X is the number of CCF nucleic acid fragments having a length within a first selected fragment length range, and Y is the number of CCF nucleic acid fragments having a length within a second selected fragment length range. In some embodiments, a first selected fragment length range is about 1 base to about 140 bases and the second selected fragment length range is about 141 bases and above. In some embodiments, a first selected fragment length range is about 1 base to about 150 bases and the second selected fragment length range is about 151 bases and above. In some embodiments, a first selected fragment length range is about 1 base to about 160 bases and the second selected fragment length range is about 161 bases and above. In some embodiments, the first selected fragment length range is about 60 bases to about 170 bases. In some embodiments, the first selected fragment length range is about 70 bases to about 160 bases. In some embodiments, the first selected fragment length range is about 75 bases to about 155 bases. In some embodiments, the first selected fragment length range is about 80 bases to about 150 bases. In some embodiments, the second selected fragment length range is about 131 bases to about 400 bases. In some embodiments, the second selected fragment length range is about 141 bases to about 350 bases. In some embodiments, the second selected fragment length range is about 146 bases to about 325 bases. In some embodiments, the second selected fragment length range is about 151 bases to about 300 bases. In some embodiments, the first selected fragment length range is about 80 bases to about 150 bases and the second selected fragment length range is about 151 bases to about 300 bases.

The term “profile” as used herein refers to a product of a mathematical and/or statistical manipulation of data that can facilitate identification of patterns and/or correlations in large quantities of data. A “profile” often includes values resulting from one or more manipulations of data or data sets, based on one or more criteria. A profile often includes multiple data points. Any suitable number of data points may be included in a profile depending on the nature and/or complexity of a data set. In certain embodiments, profiles may include 2 or more data points, 3 or more data points, 5 or more data points, 10 or more data points, 24 or more data points, 25 or more data points, 50 or more data points, 100 or more data points, 500 or more data points, 1000 or more data points, 5000 or more data points, 10,000 or more data points, or 100,000 or more data points.

In some embodiments, a profile is representative of the entirety of a data set, and in certain embodiments, a profile is representative of a part or subset of a data set. That is, a profile sometimes includes or is generated from data points representative of data that has not been filtered to remove any data, and sometimes a profile includes or is generated from data points representative of data that has been filtered to remove unwanted data. In some embodiments, a data point in a profile represents the results of data manipulation for a genomic interval (e.g., data manipulation of fragment lengths for a genomic interval). In certain embodiments, a data point in a profile includes results of data manipulation for groups of genomic intervals. In some embodiments, groups of genomic intervals may be adjacent to one another, and in certain embodiments, groups of genomic intervals may be from different parts of a chromosome or genome.

In some embodiments, a profile may be generated from data points obtained from another profile (e.g., normalized data profile renormalized to a different normalizing value to generate a renormalized data profile). In certain embodiments, a profile generated from data points obtained from another profile reduces the number of data points and/or complexity of the data set. Reducing the number of data points and/or complexity of a data set often facilitates interpretation of data and/or facilitates providing an outcome.

A profile may be a collection of normalized or non-normalized values (e.g., fragment length values; fragment length ratios) for two or more genomic intervals. A profile often includes at least one value (e.g., a fragment length value; fragment length ratio), and often comprises two or more values (e.g., a profile often has multiple fragment length values or fragment length ratios). In certain embodiments, a profile comprises one or more genomic intervals, which genomic intervals can be weighted, removed, filtered, normalized, adjusted, averaged, derived as a mean, added, subtracted, processed or transformed by any combination thereof. A profile often comprises fragment length values or fragment length ratios, where the fragment length values or fragment length ratios may be further normalized according to a suitable method. In some embodiments, fragment length values or fragment length ratios of a profile are associated with an uncertainty value.

A profile may be displayed as a plot. For example, one or more fragment length values for one or more genomic intervals can be plotted and visualized. Non-limiting examples of profile plots that can be generated include raw fragment length values, fragment length ratios, mean fragment length values, median fragment length values, average fragment length values, normalized fragment length values, z-score, p-value, principal components, the like, or combinations thereof. Profile plots allow visualization of the manipulated data, in some embodiments. In some embodiments, a profile can be generated using a static window process, and in certain embodiments, a profile can be generated using a sliding window process.

A profile generated for a test subject sometimes is compared to a profile generated for one or more reference subjects and/or samples in a training set, to facilitate interpretation of mathematical and/or statistical manipulations of a data set and/or to provide an outcome. In some embodiments, a profile is generated based on one or more starting assumptions (e.g., maternal contribution of nucleic acid (e.g., maternal fraction), fetal contribution of nucleic acid (e.g., fetal fraction), the like or combinations thereof).

### Sequence Motifs

Methods herein may comprise determining sequence motifs for nucleic acid fragment ends. A sequence motif may refer to a short pattern of bases in nucleic acid fragments (e.g., CCF nucleic acid fragments). A sequence motif can occur at an end of a fragment, and thus be part of or include a fragment end sequence. Sequence motifs for fragment ends may be any length suitable for a method described herein (e.g., 2-30 bases in length). For example, a fragment end sequence motif may be 2, 3, 4, 5, 6, 7, 8, 9, 10, 11, 12, 13, 14, 15, 16, 17, 18, 19, 20, 21, 22, 23, 24, 25, 26, 27, 28, 29, or 30 bases in length. In some embodiments, a fragment end sequence motif is three bases in length. In some embodiments, a fragment end sequence motif is four bases in length. In some embodiments, a fragment end sequence motif is five bases in length. In some embodiments, a sequence motif is a 5′ sequence motif. In some embodiments, a sequence motif is a 3′ sequence motif.

In some embodiments, sequence motifs are determined for a native end of nucleic acid fragments. In some embodiments, sequence motifs are determined for a native 5′ end of nucleic acid fragments. In some embodiments, sequence motifs are determined for a native 3′ end of nucleic acid fragments. A native end generally refers to an unmodified end of a nucleic acid fragment. In some embodiments, a native end of nucleic acid fragments is not modified in length (e.g., shortened). In some embodiments, the 5′ end of nucleic acid fragments is not modified in length (e.g., shortened). In some embodiments, the 3′ end of nucleic acid fragments is not modified in length (e.g., shortened). In this context, “not modified” means that nucleic acid fragments are isolated from a sample and processed for sequencing (e.g., processed into a sequencing library), without modifying the length of a native end of the nucleic acid fragments. For example, a nucleic acid fragment end is not shortened (e.g., it is not contacted with a restriction enzyme or nuclease or physical condition that reduces length (e.g., shearing condition, cleavage condition) to generate a non-native end). Adding one or more nucleotides for adapter ligation purposes, a phosphate, or a chemically reactive group to a native end of a nucleic acid fragment generally is not considered modifying the length of the nucleic acid fragment.

### Sequence Motif Frequency

Methods herein may comprise determining a sequence motif frequency. In some embodiments, a method herein comprises determining one or more sequence motif frequencies. In some embodiments, a method herein comprises determining a sequence motif frequency for a genome or part thereof. In some embodiments, a method herein comprises determining a sequence motif frequency for a chromosome or part thereof. In some embodiments, a method herein comprises determining one or more sequence motif frequencies for one or more chromosomes. For example, a sequence motif frequency may be determined according to the frequencies of one or more sequence motifs in the mapped sequence reads for one or more chromosomes. In some embodiments, a method herein comprises determining one or more sequence motif frequencies for one or more sequence motifs chosen from any of the 256 possible four-bp motifs (four base positions with four base possibilities at each position). In some embodiments, a method herein comprises determining one or more sequence motif frequencies for one or more sequence motifs chosen from GGAA, AGAA, GTTT, GAAT, and GGTT.

In certain embodiments, motif frequencies are processed or manipulated by a suitable method, operation or mathematical process known in the art. A motif frequency can be determined by a suitable method, operation or mathematical process. In certain embodiments, a motif frequency is derived from sequence reads where some or all of the sequence reads are weighted, removed, filtered, normalized, adjusted, averaged, derived as a mean, added, or subtracted or processed by a combination thereof. In certain embodiments, a motif frequency is derived from sequence motifs where some or all of the sequence motifs are weighted, removed, filtered, normalized, adjusted, averaged, derived as a mean, added, or subtracted or processed by a combination thereof. In some embodiments, a motif frequency is derived from raw sequence reads and or filtered sequence reads. In some embodiments, a motif frequency is derived from raw sequence motifs and or filtered sequence motifs. In certain embodiments, a motif frequency is determined by a mathematical process. In certain embodiments, a motif frequency is an average, mean or sum of sequence motifs. In some embodiments, a motif frequency is associated with an uncertainty value (e.g., a calculated variance, an error, standard deviation, Z-score, p-value, mean absolute deviation, etc.). A value for deviation can be used in place of an uncertainty value, and non-limiting examples of measures of deviation include standard deviation, average absolute deviation, median absolute deviation, standard score (e.g., Z-score, Z-score, normal score, standardized variable) and the like.

In some embodiments, motif frequencies can be manipulated or transformed (e.g., normalized, combined, added, filtered, selected, averaged, derived as a mean, the like, or a combination thereof). In some embodiments, motif frequencies can be transformed to produce normalized motif frequencies. In some embodiments, motif frequencies are normalized. In some embodiments, motif frequencies are normalized by total frequency. In some embodiments, a normalized motif diversity score (MDS) is generated. In some embodiments, a normalized motif diversity score (MDS) is generated according to the following equation:

$$MDS = {\sum }_{i = 1}^{256} - {P}_{i} * log ( {P}_{i} ) / log ( 256 )$$

### Statistical Models and Model Parameters

A method described herein may comprise estimating a fraction of fetal nucleic acid in a test sample according to one or more model parameters obtained from one or more models. A method described herein may comprise applying one or more model parameters from one or more models to one or more fragment length profiles for a test sample. A method described herein may comprise applying one or more model parameters from one or more models to one or more sequence motif frequencies for a test sample. A method described herein may comprise applying one or more model parameters from one or more models to i) one or more fragment length profiles for a test sample, and ii) one or more sequence motif frequencies for a test sample.

In some embodiments, a method described herein may comprise applying one or more model parameters from one or more models to one or more sequence read coverage features for a test sample. For example, model parameters obtained from a model that utilizes genome-wide sequence read coverage to predict fetal fraction may be applied to one or more sequence read coverage features for a test sample as described, for example, in U.S. Pat. No. 10,622,094 and in Kim et al. 2015 Prenatal Diagnosis 35:1-6, each of which is incorporated by reference in its entirety. Sequence read coverage features may include mapped sequence read quantifications for one or more genomic loci, genomic portions, genomic sections, genomic regions, genomic partitions, and the like. In some embodiments, a method described herein may comprise applying one or more model parameters from one or more models to i) one or more fragment length profiles for a test sample, and ii) one or more sequence read coverage features for a test sample. In some embodiments, a method described herein may comprise applying one or more model parameters from one or more models to i) one or more sequence motif frequencies for a test sample, and ii) one or more sequence read coverage features for a test sample. In some embodiments, a method described herein may comprise applying one or more model parameters from one or more models to i) one or more fragment length profiles for a test sample, ii) one or more sequence motif frequencies for a test sample, and iii) one or more sequence read coverage features for a test sample.

A model parameter may include values for one or more measured features described herein (e.g., fragment length profile, motif frequency) and a known or measured fetal fraction value for one or samples (e.g., one or more samples in a training set). In some embodiments, values for one or more measured features described herein (e.g., fragment length profile, motif frequency) may be determined from aggregate values as determined for a sample (e.g., a training sample) for which fetal fraction is known. Model parameters may be defined in a variety of ways, for example as discrete values or as a model function (e.g., a model curve). A model function may be derived from one or more additional mathematical transformations of one or more model parameters.

In some embodiments, a model parameter is a coefficient or constant that, in part, describes and/or defines a relation between fetal fraction and one or more measured features described herein (e.g., fragment length profile, motif frequency). In some embodiments, a model parameter is determined according to a relation for multiple fetal fractions and multiple measured features described herein (e.g., multiple fragment length profiles, multiple motif frequencies). A relation may be defined by one or more model parameters and one or more model parameters may be determined from a relation. In some embodiments, a model parameter is determined from a fitted relation according to (i) a fraction of fetal nucleic acid for each of multiple samples, and (ii) one or more measured features for multiple samples.

A model parameter can be any suitable coefficient, estimated coefficient or constant derived from a suitable statistical model and/or relation (e.g., a suitable mathematical relation, an algebraic relation, a fitted relation, a regression, a regression analysis, a regression model). A model parameter can be determined according to, derived from, or estimated from a suitable relation. In some embodiments, a model parameter is an estimated coefficient from a fitted relation. Fitting a relation for multiple samples (e.g., multiple samples in a training set) is sometimes referred to herein as training a model. Any suitable model and/or method of fitting a relationship (e.g., training a model to a training set) can be used. Non-limiting examples of a suitable model that can be used include a regression model, linear regression model, simple regression model, ordinary least squares regression model, multiple regression model, general multiple regression model, polynomial regression model, general linear model, generalized linear model, discrete choice regression model, logistic regression model, multinomial logit model, mixed logit model, probit model, multinomial probit model, ordered logit model, ordered probit model, Poisson model, multivariate response regression model, multilevel model, fixed effects model, random effects model, mixed model, nonlinear regression model, nonparametric model, semiparametric model, robust model, quantile model, isotonic model, principal components model, least angle model, local model, segmented model, errors-in-variables model, and combinations thereof. In some embodiments, a fitted relation is not a regression model. In some embodiments, a fitted relation is chosen from a decision tree model, support-vector machine model, and neural network model. The result of training a model (e.g., a regression model, a relation) is often a relation that can be described mathematically where the relation comprises one or more coefficients (e.g., model parameters). For example, for a linear least squared model, a general multiple regression model can be trained using fetal fraction values and one or more measured features described herein (e.g., fragment length profile, motif frequency) resulting in a relationship. More complex multivariate models may determine one, two, three or more model parameters. In some embodiments, a model is trained according to fetal fraction and two or more measured features described herein (e.g., fragment length profile, motif frequency) obtained from multiple samples (e.g., fitted relationships fitted to multiple samples, e.g., by a matrix).

In some embodiments, a relationship is a geometric and/or graphical relationship. The terms “relationship” and “relation”, as used herein, are synonymous. In some embodiments, a relationship is a mathematical relationship. In some embodiments, a relationship is plotted. In some embodiments, a relationship is a linear relationship. In certain embodiments, a relationship is a non-linear relationship. In certain embodiments, a relationship is a regression (e.g., a regression line). A regression can be a linear regression or a non-linear regression. A relationship can be expressed by a mathematical equation. Often a relationship is defined, in part, by one or more constants and/or one or more variables. A relationship can be generated by a method known in the art. A relationship in two dimensions can be generated for one or more samples, in certain embodiments, and a variable probative of error, or possibly probative of error, can be selected for one or more of the dimensions. A relationship can be generated, for example, using graphing software known in the art that plots a graph using values of two or more variables provided by a user. A relationship can be fitted using a method known in the art (e.g., by performing a regression, a regression analysis, e.g., by a suitable regression program, e.g., software). Certain relationships can be fitted by linear regression, and the linear regression can generate a slope value and intercept value. Certain relationships sometimes are not linear and can be fitted by a non-linear function, such as a parabolic, hyperbolic or exponential function (e.g., a quadratic function), for example.

A model parameter can be derived from a suitable relation (e.g., a suitable mathematical relation, an algebraic relation, a fitted relation, a regression, a regression analysis, a regression model) by a suitable method. In some embodiments, fitted relations are fitted by an estimation, non-limiting examples of which include least squares, ordinary least squares, linear, partial, total, generalized, weighted, non-linear, iteratively reweighted, ridge regression, least absolute deviations, Bayesian, Bayesian multivariate, reduced-rank, LASSO, Weighted Rank Selection Criteria (WRSC), Rank Selection Criteria (RSC), an elastic net estimator (e.g., an elastic net regression) and combinations thereof.

Model parameters may be obtained from any suitable sample or group of samples. In some embodiments, model parameters are obtained from a training set of samples. In some embodiments, the fraction of fetal nucleic acid is known for each sample in a training set of samples. A training set of samples may include multiple reference samples. A training set of samples may include euploid samples, aneuploid samples, or a combination of euploid samples and aneuploid samples. A training set of samples may include female samples (from pregnant subjects carrying female fetuses), male samples (from pregnant subjects carrying male fetuses), or a combination of female samples and male samples.

In some embodiments, a model parameter is determined according to one or more samples (e.g., a training set of samples). In some embodiments, a model parameter is determined according to a relation for fetal fraction (e.g., sample-specific fetal fraction) for multiple samples and one or more measured features (fragment length profile, motif frequency) determined according to multiple samples. Model parameters are often determined from multiple samples, for example, from about 20 to about 100,000 or more, from about 100 to about 100,000 or more, from about 500 to about 100,000 or more, from about 1000 to about 100,000 or more, or from about 10,000 to about 100,000 or more samples. In some embodiments, model parameters are determined from about 1,000 samples to about 2,000 samples. Model parameters can be determined from samples that are euploid (e.g., samples from subjects bearing a euploid fetus, e.g., samples where no aneuploid chromosome is present). In some embodiments, model parameters are obtained from samples comprising an aneuploid chromosome (e.g., samples from subjects bearing an aneuploid fetus). In some embodiments, model parameters are determined from multiple samples from subjects bearing a euploid fetus and from subjects bearing a trisomy fetus. Model parameters can be derived from multiple samples where the samples are from subjects bearing a male fetus and/or a female fetus.

A fetal fraction estimate can be determined for a sample (e.g., a test sample) by applying one or more model parameters to one or more measured features (e.g., fragment length profile, motif frequency) for the test sample. Applying one or more model parameters can comprise adjusting, converting and/or transforming one or more measured features (e.g., fragment length profile, motif frequency) according to a model parameter by applying any suitable mathematical manipulation, non-limiting examples of which include multiplication, division, addition, subtraction, integration, symbolic computation, algebraic computation, algorithm, trigonometric or geometric function, transformation (e.g., a Fourier transform), the like or combinations thereof. Applying one or more model parameters can comprise adjusting, converting and/or transforming one or more measured features (e.g., fragment length profile, motif frequency) according to suitable mathematical model

In some embodiments, one or more model parameters are obtained from a training set according to a fitted relation between 1) a fraction of fetal nucleic acid for each sample in a training set of samples and 2) one or more fragment length profiles for each sample in a training set of samples. In some embodiments, one or more model parameters are obtained from a training set according to a fitted relation between 1) a fraction of fetal nucleic acid for each sample in a training set of samples and 2) one or more sequence motif frequencies for each sample in a training set of samples. In some embodiments, one or more model parameters are obtained from a training set according to a fitted relation between 1) a fraction of fetal nucleic acid for each sample in a training set of samples and 2) one or more fragment length profiles and one or more sequence motif frequencies for each sample in a training set of samples.

In some embodiments, a model comprises linear regression (e.g., a linear regression for a training set of samples). In some embodiments, estimating a fraction of fetal nucleic acid for a test sample comprises applying a regression coefficient from a linear regression to one or more measured features of the test sample. In some embodiments, estimating a fraction of fetal nucleic acid for a test sample comprises applying a regression coefficient from a linear regression to one or more fragment length profiles for a test sample. In some embodiments, estimating a fraction of fetal nucleic acid for a test sample comprises applying a regression coefficient from a linear regression to one or more sequence motif frequencies for a test sample. In some embodiments, estimating a fraction of fetal nucleic acid for a test sample comprises applying a regression coefficient from a linear regression to i) one or more fragment length profiles for a test sample, and ii) one or more sequence motif frequencies for a test sample.

In some embodiments, a model comprises an Elastic net model (e.g., an Elastic net model for a training set of samples). Generally, Elastic net is a penalized linear regression model that includes both the L1 and L2 penalties during training. A hyperparameter “alpha” is provided to assign how much weight is given to each of the L1 and L2 penalties. In some embodiments, estimating a fraction of fetal nucleic acid for a test sample comprises applying a coefficient from an Elastic net model to one or more measured features of the test sample. In some embodiments, estimating a fraction of fetal nucleic acid for a test sample comprises applying a coefficient from an Elastic net model to one or more fragment length profiles for a test sample. In some embodiments, estimating a fraction of fetal nucleic acid for a test sample comprises applying a coefficient from an Elastic net model to one or more sequence motif frequencies for a test sample. In some embodiments, estimating a fraction of fetal nucleic acid for a test sample comprises applying a coefficient from an Elastic net model to i) one or more fragment length profiles for a test sample, and ii) one or more sequence motif frequencies for a test sample.

In some embodiments, a model comprises an XGBoost model (e.g., an XGBoost model for a training set of samples). Generally, XGBoost is an efficient open-source implementation of the gradient boosted trees algorithm. Gradient boosting is a supervised learning algorithm, which attempts to accurately predict a target variable by combining the estimates of a set of simpler, weaker models. In some embodiments, estimating a fraction of fetal nucleic acid for a test sample comprises applying a coefficient from an XGBoost model to one or more measured features of the test sample. In some embodiments, estimating a fraction of fetal nucleic acid for a test sample comprises applying a coefficient from an XGBoost model to one or more fragment length profiles for a test sample. In some embodiments, estimating a fraction of fetal nucleic acid for a test sample comprises applying a coefficient from an XGBoost model to one or more sequence motif frequencies for a test sample. In some embodiments, estimating a fraction of fetal nucleic acid for a test sample comprises applying a coefficient from an XGBoost model to i) one or more fragment length profiles for a test sample, and ii) one or more sequence motif frequencies for a test sample. A fetal fraction (e.g., for a test sample) can be determined according to multiple fetal fraction sub-estimates (e.g., for the same test sample), in certain applications, by any suitable method. In some embodiments, a method for increasing the accuracy of the estimation of a fraction of fetal nucleic acid in a test sample from a pregnant female comprises determining one or more fetal fraction sub-estimates where the estimate of fetal fraction for the sample is determined according to the one or more fetal fraction sub-estimates. In some embodiments, estimating or determining a fraction of fetal nucleic acid for a sample (e.g., a test sample) comprises summing one or more fetal fraction sub-estimates. Summing can comprise determining an average, mean, median, AUC, or integral value according to multiple fetal fraction sub-estimates. Fetal fraction sub-estimates may be derived from a subset of measured features for the test sample (e.g., a subset of fragment length profiles and/or a subset of motif frequencies). In some embodiments, a first subset includes one or more fragment length profiles for the test sample, and a second subset includes one or more motif frequencies for the test sample.

### Data Processing

Measured features for a test sample and/or measured features for samples in a training set may include fragment lengths, fragment length ratios, fragment length profiles, motif counts, and motif frequencies, and may be referred to herein as raw data, since the data represents unmanipulated quantifications of such features. In some embodiments, measured feature data in a data set can be processed further (e.g., mathematically and/or statistically manipulated) and/or displayed to facilitate providing an outcome. In certain embodiments, data sets, including larger data sets, may benefit from pre-processing to facilitate further analysis. Pre-processing of data sets sometimes involves removal of redundant and/or uninformative data. Without being limited by theory, data processing and/or preprocessing may (i) remove noisy data, (ii) remove uninformative data, (iii) remove redundant data, (iv) reduce the complexity of larger data sets, and/or (v) facilitate transformation of the data from one form into one or more other forms. The terms “pre-processing” and “processing” when utilized with respect to data or data sets are collectively referred to herein as “processing”. Processing can render data more amenable to further analysis, and can generate an outcome in some embodiments. In some embodiments, one or more or all processing methods are performed by a processor, a micro-processor, a computer, in conjunction with memory and/or by a microprocessor-controlled machine.

The term “noisy data” as used herein refers to (a) data that has a significant variance between data points when analyzed or plotted, (b) data that has a significant standard deviation (e.g., greater than 3 standard deviations), (c) data that has a significant standard error of the mean, the like, and combinations of the foregoing. Noisy data sometimes occurs due to the quantity and/or quality of starting material (e.g., nucleic acid sample), and sometimes occurs as part of processes for preparing or replicating DNA used to generate sequence reads. In certain embodiments, noise results from certain sequences being over represented when prepared using PCR-based methods.

The term “uninformative data” as used herein refer to data having a numerical value that is significantly different from a predetermined threshold value or falls outside a predetermined cutoff range of values. A threshold value or range of values often is calculated by mathematically and/or statistically manipulating data (e.g., from a reference and/or subject). In some embodiments, an uncertainty value is determined. An uncertainty value generally is a measure of variance or error and can be any suitable measure of variance or error. In some embodiments, an uncertainty value is a standard deviation, standard error, calculated variance, p-value, or mean absolute deviation (MAD).

Any suitable procedure can be utilized for processing data sets described herein. Non-limiting examples of procedures suitable for use for processing data sets include filtering, normalizing, weighting, monitoring peak heights, monitoring peak areas, monitoring peak edges, determining area ratios, mathematical processing of data, statistical processing of data, application of statistical algorithms, analysis with fixed variables, analysis with optimized variables, plotting data to identify patterns or trends for additional processing, the like and combinations of the foregoing. In some embodiments, data sets are processed based on various features (e.g., GC content, redundant mapped reads, centromere regions, telomere regions, the like and combinations thereof) and/or variables (e.g., fetal gender, maternal age, maternal ploidy, percent contribution of fetal nucleic acid, the like or combinations thereof). In certain embodiments, processing data sets as described herein can reduce the complexity and/or dimensionality of large and/or complex data sets. In some embodiments, data sets can include from thousands to millions of sequence reads, fragment lengths, fragment length ratios, fragment length profiles, sequence motifs, motif counts, and motif frequencies for each test sample and/or each sample in a training set.

Data processing can be performed in any number of steps, in certain embodiments. For example, data may be processed using only a single processing procedure, in some embodiments, and in certain embodiments, data may be processed using 1 or more, 5 or more, 10 or more or 20 or more processing steps (e.g., 1 or more processing steps, 2 or more processing steps, 3 or more processing steps, 4 or more processing steps, 5 or more processing steps, 6 or more processing steps, 7 or more processing steps, 8 or more processing steps, 9 or more processing steps, 10 or more processing steps, 11 or more processing steps, 12 or more processing steps, 13 or more processing steps, 14 or more processing steps, 15 or more processing steps, 16 or more processing steps, 17 or more processing steps, 18 or more processing steps, 19 or more processing steps, or 20 or more processing steps). In some embodiments, processing steps may be the same step repeated two or more times, and in certain embodiments, processing steps may be two or more different processing steps, carried out simultaneously or sequentially. In some embodiments, any suitable number and/or combination of the same or different processing steps can be utilized to process sequence read data to facilitate providing an outcome.

In certain embodiments, processing data sets by the criteria described herein may reduce the complexity and/or dimensionality of a data set. For example, a principal component analysis (PCA) can reduce the complexity and/or dimensionality of a data set. A PCA can be performed by a suitable PCA method, or a variation thereof. Non-limiting examples of a PCA method include a canonical correlation analysis (CCA), a Karhunen-Loève transform (KLT), a Hotelling transform, a proper orthogonal decomposition (POD), a singular value decomposition (SVD) of X, an eigenvalue decomposition (EVD) of XTX, a factor analysis, an Eckart-Young theorem, a Schmidt-Mirsky theorem, empirical orthogonal functions (EOF), an empirical eigenfunction decomposition, an empirical component analysis, quasiharmonic modes, a spectral decomposition, an empirical modal analysis, the like, variations or combinations thereof. A PCA often identifies one or more principal components. In some embodiments, a PCA identifies a 1^{st}, 2^{nd}, 3^{rd}, 4^{th}, 5^{th}, 6^{th}, 7^{th}, 8^{th}, 9^{th}, and a 10^{th} or more principal components.

In some embodiments, one or more processing steps can comprise one or more normalization steps. Normalization can be performed by a suitable method described herein or known in the art. In certain embodiments, normalization comprises adjusting values measured on different scales to a notionally common scale. In certain embodiments, normalization comprises a sophisticated mathematical adjustment to bring probability distributions of adjusted values into alignment. In some embodiments, normalization comprises aligning distributions to a normal distribution. In certain embodiments, normalization comprises mathematical adjustments that allow comparison of corresponding normalized values for different datasets in a way that eliminates the effects of certain gross influences (e.g., error and anomalies). In certain embodiments, normalization comprises scaling. Normalization sometimes comprises division of one or more data sets by a predetermined variable or formula.

Any suitable number of normalizations can be used. In some embodiments, data sets can be normalized 1 or more, 5 or more, 10 or more or even 20 or more times. Data sets can be normalized to values (e.g., normalizing value) representative of any suitable feature or variable (e.g., sample data, reference data, or both). Normalizing a data set sometimes has the effect of isolating statistical error, depending on the feature or property selected as the predetermined normalization variable. Normalizing a data set sometimes also allows comparison of data characteristics of data having different scales, by bringing the data to a common scale (e.g., predetermined normalization variable). In some embodiments, one or more normalizations to a statistically derived value can be utilized to minimize data differences and diminish the importance of outlying data.

In some embodiments, a processing step comprises a weighting. The terms “weighted”, “weighting” or “weight function” or grammatical derivatives or equivalents thereof, as used herein, refer to a mathematical manipulation of a portion or all of a data set sometimes utilized to alter the influence of certain data set features or variables with respect to other data set features or variables. A weighting function can be used to increase the influence of data with a relatively small measurement variance, and/or to decrease the influence of data with a relatively large measurement variance, in some embodiments. A non-limiting example of a weighting function is [1/(standard deviation)²]. A weighting step sometimes is performed in a manner substantially similar to a normalizing step. In some embodiments, a data set is divided by a predetermined variable (e.g., weighting variable). A predetermined variable (e.g., minimized target function, Phi) often is selected to weigh different parts of a data set differently (e.g., increase the influence of certain data types while decreasing the influence of other data types).

In certain embodiments, a processing step can comprise one or more mathematical and/or statistical manipulations. Any suitable mathematical and/or statistical manipulation, alone or in combination, may be used to analyze and/or manipulate a data set described herein. Any suitable number of mathematical and/or statistical manipulations can be used. In some embodiments, a data set can be mathematically and/or statistically manipulated 1 or more, 5 or more, 10 or more or 20 or more times. Non-limiting examples of mathematical and statistical manipulations that can be used include addition, subtraction, multiplication, division, algebraic functions, least squares estimators, curve fitting, differential equations, rational polynomials, double polynomials, orthogonal polynomials, z-scores, p-values, chi values, phi values, analysis of peak levels, determination of peak edge locations, calculation of peak area ratios, analysis of median chromosomal level, calculation of mean absolute deviation, sum of squared residuals, mean, standard deviation, standard error, the like or combinations thereof. Non-limiting examples of data set variables or features that can be statistically manipulated include fragment lengths, fragment length ratios, fragment length profiles, motif counts, motif frequencies, fetal fraction, the like or combinations thereof.

In some embodiments, a processing step can comprise the use of one or more statistical algorithms. Any suitable statistical algorithm, alone or in combination, may be used to analyze and/or manipulate a data set described herein. Any suitable number of statistical algorithms can be used. In some embodiments, a data set can be analyzed using 1 or more, 5 or more, 10 or more or 20 or more statistical algorithms. Non-limiting examples of statistical algorithms suitable for use with methods described herein include decision trees, counternulls, multiple comparisons, omnibus test, Behrens-Fisher problem, bootstrapping, Fisher's method for combining independent tests of significance, null hypothesis, type I error, type II error, exact test, one-sample Z test, two-sample Z test, one-sample t-test, paired t-test, two-sample pooled t-test having equal variances, two-sample unpooled t-test having unequal variances, one-proportion z-test, two-proportion z-test pooled, two-proportion z-test unpooled, one-sample chi-square test, two-sample F test for equality of variances, confidence interval, credible interval, significance, meta analysis, simple linear regression, robust linear regression, the like or combinations of the foregoing. Non-limiting examples of data set variables or features that can be analyzed using statistical algorithms include fragment lengths, fragment length ratios, fragment length profiles, motif counts, motif frequencies, fetal fraction, the like or combinations thereof.

In certain embodiments, a data set can be analyzed by utilizing multiple (e.g., 2 or more) statistical algorithms (e.g., least squares regression, principle component analysis, linear discriminant analysis, quadratic discriminant analysis, bagging, neural networks, support vector machine models, random forests, classification tree models, K-nearest neighbors, logistic regression and/or loss smoothing) and/or mathematical and/or statistical manipulations (e.g., referred to herein as manipulations). The use of multiple manipulations can generate an N-dimensional space that can be used to provide an outcome, in some embodiments. In certain embodiments, analysis of a data set by utilizing multiple manipulations can reduce the complexity and/or dimensionality of the data set. For example, the use of multiple manipulations on a training data set can generate an N-dimensional space (e.g., probability plot) that can be used to represent fetal fraction. Analysis of test samples using a substantially similar set of manipulations can be used to generate an N-dimensional point for each of the test samples. The complexity and/or dimensionality of a test subject data set sometimes is reduced to a single value or N-dimensional point that can be readily compared to the N-dimensional space generated from the training data.

In some embodiments, data processing includes cross-validation. Cross-validation, sometimes is referred to as rotation estimation. In some embodiments, a cross-validation approach is applied to assess how accurately a predictive model will perform in practice using a test sample. In some embodiments, one round of cross-validation comprises partitioning a sample of data into complementary subsets, performing a cross validation analysis on one subset (e.g., sometimes referred to as a training set), and validating the analysis using another subset (e.g., sometimes called a validation set or test set). In certain embodiments, multiple rounds of cross-validation are performed using different partitions and/or different subsets). Non-limiting examples of cross-validation approaches include leave-one-out, sliding edges, K-fold, 2-fold, repeat random sub-sampling, the like or combinations thereof. In some embodiments, a cross-validation randomly selects a work set containing 80% of a set of samples with known fetal fractions and uses that subset to train a model. In certain embodiments, the random selection is repeated multiple times.

### Machines, Software and Interfaces

Certain processes and methods described herein (e.g., obtaining sequence reads, mapping sequence reads, measuring nucleic acid fragment lengths, generating nucleic acid fragment length profiles, identifying sequence motifs, determining sequence motif frequencies, estimating fetal fraction, and the like) often cannot be performed in the human mind and thus cannot be performed without a computer, microprocessor, software, module, or other machine. Methods described herein typically are computer-implemented methods, and one or more portions of a method sometimes are performed by one or more processors (e.g., microprocessors), computers, or microprocessor-controlled machines. Embodiments pertaining to methods described in this document generally are applicable to the same or related processes implemented by instructions in systems, machines and computer program products described herein. Embodiments pertaining to methods described in this document generally can be applicable to the same or related processes implemented by a non-transitory computer-readable storage medium with an executable program stored thereon, where the program instructs a microprocessor to perform the method, or a part thereof. In some embodiments, processes and methods described herein are performed by automated methods. In some embodiments, one or more steps and a method described herein is carried out by a microprocessor and/or computer, and/or carried out in conjunction with memory. In some embodiments, an automated method is embodied in software, modules, microprocessors, peripherals and/or a machine comprising the like, that determine sequence reads, counts, mapped sequence reads, fragment lengths, levels, profiles, sequence motifs, sequence motif frequencies, fetal fraction, normalizations, comparisons, range setting, categorization, adjustments, plotting, outcomes, transformations and identifications. As used herein, software refers to computer readable program instructions that, when executed by a microprocessor, perform computer operations, as described herein.

Sequence reads, fragment lengths, profiles, and/or sequence motif frequencies derived from a test subject (e.g., a patient, a pregnant subject) and/or from a reference subject can be further analyzed and processed to estimate a fraction of fetal nucleic acid in a sample from a test subject or reference subject. Sequence reads, fragment lengths, profiles, and/or sequence motif frequencies sometimes are referred to as “data” or “data sets”. In some embodiments, data or data sets can be characterized by one or more features or variables (e.g., sequence based [e.g., GC content, specific nucleotide sequence, the like], function specific [e.g., expressed genes, cancer genes, the like], location based [genome-specific, chromosome-specific, portion or portion-specific], the like and combinations thereof). In certain embodiments, data or data sets can be organized into a matrix having two or more dimensions based on one or more features or variables. Data organized into matrices can be organized using any suitable features or variables. A non-limiting example of data in a matrix includes data that is organized by maternal age, maternal ploidy, and fetal contribution.

Machines, software and interfaces may be used to conduct methods described herein. Using machines, software and interfaces, a user may enter, request, query or determine options for using particular information, programs or processes (e.g., mapping sequence reads, processing mapped data and/or providing an outcome), which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by a suitable hardware media (e.g., flash drive), and/or a user may send a data set from one system to another for subsequent processing and/or providing an outcome (e.g., send sequence read data from a sequencer to a computer system for sequence read mapping; send mapped sequence data to a computer system for processing and yielding an outcome and/or report).

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A system sometimes comprises a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to measure fragment lengths, generate fragment length profiles, determine sequence motifs, determine sequence motif frequencies, and/or estimate a fraction of fetal nucleic acid in a test sample from the sequence reads.

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output means may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet-based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus or machine may serve as data that can be input via an input device. In certain embodiments, mapped sequence reads may serve as data that can be input via an input device. In certain embodiments, nucleic acid fragment size (e.g., length) may serve as data that can be input via an input device. In certain embodiments, sequence motifs may serve as data that can be input via an input device. In certain embodiments, a combination of nucleic acid fragment size (e.g., length) and sequence motifs may serve as data that can be input via an input device. In certain embodiments, simulated data is generated by an in-silico process and the simulated data serves as data that can be input via an input device. The term “in silico” refers to research and experiments performed using a computer. In silico processes include, but are not limited to, mapping sequence reads and processing mapped sequence reads according to processes described herein.

A system may include software useful for performing a process described herein, and software can include one or more modules for performing such processes (e.g., sequencing module, logic processing module, data display organization module). The term “software” refers to computer readable program instructions that, when executed by a computer, perform computer operations. Instructions executable by the one or more microprocessors sometimes are provided as executable code, that when executed, can cause one or more microprocessors to implement a method described herein. A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can comprise a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g., frequencies, audible or non- audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to an machine, peripheral, component or another module. A module can perform one or more of the following non- limiting functions: obtaining sequence reads, mapping sequence reads, measuring nucleic acid fragment lengths, generating nucleic acid fragment length profiles, identifying sequence motifs, determining sequence motif frequencies, estimating fetal fraction, normalizing, providing a normalized profile, providing a normalized sequence motif frequency, comparing two or more profiles, comparing two or more sequence motif frequencies, providing uncertainty values, providing or determining expected ranges, providing adjustments, categorizing, plotting, and/or determining an outcome, for example. A microprocessor can, in certain embodiments, carry out the instructions in a module. In some embodiments, one or more microprocessors are required to carry out instructions in a module or group of modules. A module can provide data and/or information to another module, machine or source and can receive data and/or information from another module, machine or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules may be located in different machines in different physical locations.

A machine, in some embodiments, comprises at least one microprocessor for carrying out the instructions in a module. Sequence reads mapped to a reference genome sometimes are accessed by a microprocessor that executes instructions configured to carry out a method described herein. Sequence reads that are accessed by a microprocessor can be within memory of a system, and the sequence reads can be accessed and placed into the memory of the system after they are obtained. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a module. In certain embodiments a machine comprises one or more modules. A machine comprising a module often can receive and transfer one or more of data and/or information to and from other modules. In certain embodiments, a machine comprises peripherals and/or components. In certain embodiments a machine can comprise one or more peripherals or components that can transfer data and/or information to and from other modules, peripherals and/or components. In certain embodiments a machine interacts with a peripheral and/or component that provides data and/or information. In certain embodiments peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like,), the world wide web (www), the internet, a computer and/or another module.

Software often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash drives, RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a module that specifically obtains or receives data (e.g., a data receiving module that receives sequence read data and/or mapped read data) and may include a module that specifically processes the data (e.g., a processing module that processes received data). The terms “obtaining” and “receiving” input information refers to receiving data (e.g., sequence reads, mapped reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

In some embodiments, provided are computer program products, such as, for example, a computer program product comprising a computer usable medium having a computer readable program code embodied therein, the computer readable program code adapted to be executed to implement a method comprising: a) obtaining sequence reads mapped to a reference genome, where the sequence reads are reads of circulating cell-free (CCF) nucleic acid from a test sample from a pregnant subject; b) measuring fragment lengths for a plurality of circulating cell-free nucleic acid fragments; c) generating one or more fragment length profiles for the test sample; d) determining a sequence motif for a plurality of circulating cell-free nucleic acid fragment ends; e) determining one or more sequence motif frequencies for the test sample; f) estimating a fraction of fetal nucleic acid for the test sample according to i) the one or more fragment length profiles for the test sample, and ii) the one or more sequence motif frequencies for the test sample.

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In some embodiments, an algorithm can be configured or modified to include margin of errors, statistical analysis, statistical significance, and/or comparison to other information or data sets (e.g., applicable when using a neural net or clustering algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. In some embodiments, simulated data includes hypothetical various samplings of different groupings of sequence reads. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of an identified results, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more microprocessors in certain embodiments. A microprocessor can be connected to a communication bus. A computer system may include a main memory, often random-access memory (RAM), and can also include a secondary memory. Memory in some embodiments, comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for estimating a fraction of fetal nucleic acid.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

One entity can generate counts of sequence reads, map the sequence reads to a reference genome, and utilize the mapped reads in a method, system, machine, apparatus or computer program product described herein, in some embodiments. Sequence reads mapped to a reference genome sometimes are transferred by one entity to a second entity for use by the second entity in a method, system, machine, apparatus or computer program product described herein, in certain embodiments.

In some embodiments, one entity generates sequence reads and a second entity maps those sequence reads to a reference genome. The second entity sometimes utilizes the mapped reads in a method, system, machine or computer program product described herein. In certain embodiments the second entity transfers the mapped reads to a third entity, and the third entity utilizes the mapped reads in a method, system, machine or computer program product described herein. In embodiments involving a third entity, the third entity sometimes is the same as the first entity. That is, the first entity sometimes transfers sequence reads to a second entity, which second entity can map sequence reads to a reference genome, and the second entity can transfer the mapped reads to a third entity. A third entity sometimes can utilize the mapped reads in a method, system, machine or computer program product described herein, where the third entity sometimes is the same as the first entity, and sometimes the third entity is different from the first or second entity. In some embodiments, one entity obtains blood from a pregnant subject, optionally isolates nucleic acid from the blood (e.g., from the plasma or serum), and transfers the blood or nucleic acid to a second entity that generates sequence reads from the nucleic acid.

Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like. Any type of computer-readable media that can store data that is accessible by a computer, such as magnetic cassettes, flash memory cards, digital video disks, Bernoulli cartridges, random access memories (RAMs), read only memories (ROMs), and the like, may be used in the operating environment.

Provided herein, in certain embodiments, is a system comprising one or more microprocessors and memory, which memory comprises instructions executable by the one or more microprocessors and which memory comprises sequence reads mapped to a reference genome, which sequence reads are reads of circulating cell-free (CCF) nucleic acid from a test sample from a pregnant subject, and which instructions executable by the one or more microprocessors are configured to a) measure fragment lengths for a plurality of circulating cell-free nucleic acid fragments; b) generate one or more fragment length profiles for the test sample; c) determine a sequence motif for a plurality of circulating cell-free nucleic acid fragment ends; d) determine one or more sequence motif frequencies for the test sample; and e) estimate a fraction of fetal nucleic acid for the test sample according to i) the one or more fragment length profiles for the test sample, and ii) the one or more sequence motif frequencies for the test sample.

Also provided herein, in certain embodiments, is a machine comprising one or more microprocessors and memory, which memory comprises instructions executable by the one or more microprocessors and which memory comprises sequence reads mapped to a reference genome, which sequence reads are reads of circulating cell-free nucleic acid from a test sample from a pregnant subject, and which instructions executable by the one or more microprocessors are configured to a) measure fragment lengths for a plurality of circulating cell-free nucleic acid fragments; b) generate one or more fragment length profiles for the test sample; c) determine a sequence motif for a plurality of circulating cell-free nucleic acid fragment ends; d) determine one or more sequence motif frequencies for the test sample; and e) estimate a fraction of fetal nucleic acid for the test sample according to i) the one or more fragment length profiles for the test sample, and ii) the one or more sequence motif frequencies for the test sample.

Also provided herein, in certain embodiments, is a non-transitory computer-readable storage medium with an executable program stored thereon, where the program instructs a microprocessor to perform the following: a) access sequence reads mapped to a reference genome, which sequence reads are reads of circulating cell-free nucleic acid from a test sample from a pregnant subject, b) measure fragment lengths for a plurality of circulating cell-free nucleic acid fragments; c) generate one or more fragment length profiles for the test sample; d) determine a sequence motif for a plurality of circulating cell-free nucleic acid fragment ends; e) determine one or more sequence motif frequencies for the test sample; and f) estimate a fraction of fetal nucleic acid for the test sample according to i) the one or more fragment length profiles for the test sample, and ii) the one or more sequence motif frequencies for the test sample.

### Certain Implementations

Following are non-limiting examples of certain implementations of the technology.

A1. A method for estimating a fraction of fetal nucleic acid in a test sample from a pregnant subject comprising:
a) obtaining sequence reads mapped to a reference genome, wherein the sequence reads are reads of circulating cell-free (CCF) nucleic acid from a test sample from a pregnant subject;
b) measuring fragment lengths for a plurality of circulating cell-free nucleic acid fragments;
c) generating one or more fragment length profiles for the test sample;
d) determining a sequence motif for a plurality of circulating cell-free nucleic acid fragment ends;
e) determining one or more sequence motif frequencies for the test sample; and
f) estimating a fraction of fetal nucleic acid for the test sample according to i) the one or more fragment length profiles for the test sample, and ii) the one or more sequence motif frequencies for the test sample.

A2. The method of embodiment A1, wherein the sequence reds are obtained by a paired-end sequencing process and the sequence reads are paired-end sequence reads.

A3. The method of embodiment A2, wherein the fragment lengths are measured in (b) according to mapped positions of the paired-end sequence reads.

A4. The method of any one of embodiments A1-A3, wherein the fragment lengths are measured for a plurality of genomic intervals.

A5. The method of embodiment A4, wherein the genomic intervals are about 100 kilobases (kb) in length.

A6. The method of any one of embodiments A1-A5, wherein the one or more fragment length profiles are generated in (c) according to ratios of X to Y for a plurality of genomic intervals, wherein X is the number of CCF nucleic acid fragments having a length within a first selected fragment length range, and Y is the number of CCF nucleic acid fragments having a length within a second selected fragment length range.

A7. The method of embodiment A6, wherein the first selected fragment length range is about 80 bases to about 150 bases and the second selected fragment length range is about 151 bases to about 300 bases.

A8. The method of embodiment A6 or A7, wherein the genomic intervals are about 100 kilobases (kb) in length.

A9. The method of any one of embodiments A1-A8, wherein the one or more fragment length profiles are generated in (c) for one or more genomic segments.

A10. The method of embodiment A9, wherein the genomic segments are 5 megabases (Mb) in length.

A11. The method of any one of embodiments A1-A10, wherein the sequence motif in (d) is a 5′ sequence motif.

A12. The method of any one of embodiments A1-A11, wherein the sequence motif in (d) is a four-base pair (bp) sequence motif.

A13. The method of any one of embodiments A1-A12, wherein (e) comprises determining one or more sequence motif frequencies for one or more chromosomes.

A14. The method of any one of embodiments A1-A13, wherein (e) comprises determining one or more frequencies for one or more sequence motifs chosen from GGAA, AGAA, GTTT, GAAT, and GGTT.

A15. The method of any one of embodiments A1-A14, wherein the one or more sequence motif frequencies are determined according to the frequencies of one or more sequence motifs in the mapped sequence reads for one or more chromosomes.

A15. The method of any one of embodiments A1-A14, wherein estimating the fraction of fetal nucleic acid for the test sample in (f) comprises applying one or more model parameters from one or more models to i) the one or more fragment length profiles for the test sample, and ii) the one or more sequence motif frequencies for the test sample.

A16. The method of embodiment A15, wherein the model parameters are obtained from a training set of samples.

A17. The method of embodiment A16, wherein the fraction of fetal nucleic acid is known for each sample in the training set of samples.

A18. The method of any one of embodiments A17, wherein the one or more model parameters are obtained from the training set according to a fitted relation between 1) the fraction of fetal nucleic acid for each sample in the training set of samples and 2) one or more fragment length profiles for each sample in the training set of samples.

A19. The method of any one of embodiments A17 or A18, wherein the one or more model parameters are obtained from the training set according to a fitted relation between 1) the fraction of fetal nucleic acid for each sample in the training set of samples and 2) one or more sequence motif frequencies for each sample in the training set of samples.

A20. The method of any one of embodiments A17-A19, wherein the one or more model parameters are obtained from the training set according to a fitted relation between 1) the fraction of fetal nucleic acid for each sample in the training set of samples and 2) one or more fragment length profiles and one or more sequence motif frequencies for each sample in the training set of samples.

A20.1 The method of any one of any one of embodiments A15-A20, wherein the one or more model parameters comprise a coefficient derived from the one or more models.

A21. The method of any one of embodiments A15-A20.1, wherein the one or more models comprise linear regression.

A22. The method of embodiment A21, wherein estimating the fraction of fetal nucleic acid for the test sample in (f) comprises applying a regression coefficient from the linear regression to i) the one or more fragment length profiles for the test sample, and ii) the one or more sequence motif frequencies for the test sample.

A23. The method of any one of embodiments A15-A22, wherein the one or more models comprise Elastic net.

A24. The method of embodiment A23, wherein estimating the fraction of fetal nucleic acid for the test sample in (f) comprises applying a coefficient from the Elastic net model to i) the one or more fragment length profiles for the test sample, and ii) the one or more sequence motif frequencies for the test sample.

A25. The method of any one of embodiments A15-A24, wherein the one or more models comprise XGBoost.

A26. The method of embodiment A25, wherein estimating the fraction of fetal nucleic acid for the test sample in (f) comprises applying a coefficient from the XGBoost model to i) the one or more fragment length profiles for the test sample, and ii) the one or more sequence motif frequencies for the test sample.

A27. The method of any one of embodiments A1-A26, further comprising prior to (a), sequencing the circulating cell-free (CCF) nucleic acid from the test sample by a sequencing process.

A28. The method of embodiment A27, wherein the sequencing process is a non-targeted sequencing process.

A29. The method of embodiment A27, wherein the sequencing process is a massively parallel sequencing process.

A30. The method of embodiment A27, wherein the sequencing process is a non-targeted massively parallel sequencing process.

A31. The method of any one of embodiments A27-A30, wherein the circulating cell-free (CCF) nucleic acid from the test sample is sequenced at a fold coverage of 1.0 or greater.

A32. The method of any one of embodiments A27-A30, wherein the circulating cell-free (CCF) nucleic acid from the test sample is sequenced at a fold coverage of less than 1.0.

A33. The method of any one of embodiments A27-A32, wherein the sequencing process generates thousands to millions of sequence reads.

A34. The method of any one of embodiments A1-A33, further comprising prior to (a), mapping the sequence reads to the reference genome.

A35. The method of any one of embodiments A1-A33, further comprising prior to (a), mapping thousands to millions of sequence reads to the reference genome.

A36. The method of any one of embodiments A1-A35, further comprising obtaining quantifications of mapped sequence reads for the test sample.

A37. The method of embodiment A36, wherein (f) comprises estimating a fraction of fetal nucleic acid for the test sample according to i) the one or more fragment length profiles for the test sample, ii) the one or more sequence motif frequencies for the test sample, and iii) the quantifications of mapped sequence reads for the test sample.

B1. A system comprising one or more microprocessors and memory, which memory comprises instructions executable by the one or more microprocessors and which memory comprises sequence reads mapped to a reference genome, wherein the sequence reads are reads of circulating cell-free (CCF) nucleic acid from a test sample from a pregnant subject, and wherein the instructions executable by the one or more microprocessors are configured to:
a) measure fragment lengths for a plurality of circulating cell-free nucleic acid fragments;
b) generate one or more fragment length profiles for the test sample;
c) determine a sequence motif for a plurality of circulating cell-free nucleic acid fragment ends;
d) determine one or more sequence motif frequencies for the test sample; and
e) estimate a fraction of fetal nucleic acid for the test sample according to i) the one or more fragment length profiles for the test sample, and ii) the one or more sequence motif frequencies for the test sample.

B2. The system of embodiment B1 further comprising one or more features of embodiments A2-A35 and/or further configured to perform one or more methods of embodiments A2-A37.

C1. A machine comprising one or more microprocessors and memory, which memory comprises instructions executable by the one or more microprocessors and which memory comprises sequence reads mapped to a reference genome, wherein the sequence reads are reads of circulating cell-free nucleic acid from a test sample from a pregnant subject, and wherein the instructions executable by the one or more microprocessors are configured to:
a) measure fragment lengths for a plurality of circulating cell-free nucleic acid fragments;
b) generate one or more fragment length profiles for the test sample;
c) determine a sequence motif for a plurality of circulating cell-free nucleic acid fragment ends;
d) determine one or more sequence motif frequencies for the test sample; and
e) estimate a fraction of fetal nucleic acid for the test sample according to i) the one or more fragment length profiles for the test sample, and ii) the one or more sequence motif frequencies for the test sample.

C2. The machine of embodiment C1 further comprising one or more features of embodiments A2-A35 and/or further configured to perform one or more methods of embodiments A2-A37.

D1. A non-transitory computer-readable storage medium with an executable program stored thereon, where the program instructs a microprocessor to perform the following:
a) access sequence reads mapped to a reference genome, wherein the sequence reads are reads of circulating cell-free nucleic acid from a test sample from a pregnant subject,
b) measure fragment lengths for a plurality of circulating cell-free nucleic acid fragments;
c) generate one or more fragment length profiles for the test sample;
d) determine a sequence motif for a plurality of circulating cell-free nucleic acid fragment ends;
e) determine one or more sequence motif frequencies for the test sample; and
f) estimate a fraction of fetal nucleic acid for the test sample according to i) the one or more fragment length profiles for the test sample, and ii) the one or more sequence motif frequencies for the test sample.

D2. The non-transitory computer-readable storage medium of embodiment D1 further comprising one or more features of embodiments A2-A35 and/or further configured to perform one or more methods of embodiments A2-A37.

### EXAMPLES

The examples set forth below illustrate certain implementations and do not limit the technology.

### Example 1: Fetal fraction prediction with fragmentomics

In this Example, a method for predicting fetal fraction in a sample based on fragmentomics parameters (i.e., end motif frequencies and fragment length profiles) is described.

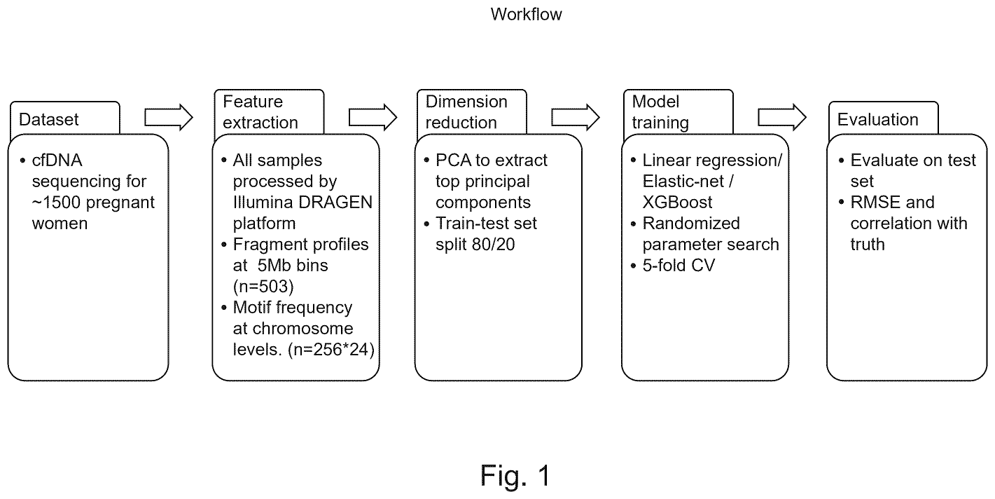

An example workflow is provided in FIG. 1. An internal dataset of cfDNA sequencing data from about 1500 pregnant women was used for the training set and test set described in this Example. The sequencing data was processed using Illumina's DRAGN 4.0 platform to obtain fragment profiles (#short fragments (80-150 bp)/#long fragments (151-300 bp)) for 5 Mb genomic bins (n=503) and motif frequencies at chromosome levels (n=256×24). 256 is the number of possible 4-bp motifs and 24 is 22 autosomes plus chromosome X and chromosome Y. Motif frequencies were normalized by total frequency and a normalized motif diversity score (MDS) was generated according to the following equation:

$$MDS = {\sum }_{i = 1}^{256} - {P}_{i} * log ( {P}_{i} ) / log ( 256 )$$

After read alignment and feature extraction, data was aggregated and summarized into a spreadsheet table as shown below:

### Fragment Profile

| | | Fragment | Fragment | | Fragnment |
|---|---|---|---|---|---|
| SampleID | Fetal fraction | profile 1 | profile 2 | . . . | profile 503 |
| 1 | 0.1 | 0.56 | . . . | | . . . |
| 2 | 0.14 | . . . | | | . . . |
| . . . | | | | | |
| N | 0.2 | . . . | | | . . . |

### Motif Frequencies

| SampleID | Fetal fraction | Motif 1 | Motif 2 | . . . | Motif 256 |
|---|---|---|---|---|---|
| 1 | 0.1 | 0.35 | . . . | | . . . |
| 2 | 0.14 | . . . | | | |
| . . . | | | | | |
| N | 0.2 | . . . | | | . . . |

A principal component analysis (PCA) was applied to the data to extract the top principal components. In particular, features were analyzed with PCA to extract the most relevant variables that predict fetal fraction. PCA on fragment profiles and motif frequency extracted first principal components that explained at least 99% variances, which reduced total variables down to 32.

The data table was then split into a training set and a test set at an 80 to 20 ratio. Three machine learning models (i.e., linear regression, Elastic net, and XGBoost) were performed on the training data set separately using the R codes below:

FF_predicted <- predict(model_linear_regression, test)
FF_predicted <- predict(model_elastic_net, test)
FF_predicted <- predict(model_xgboost, test)

The linear regression model was trained using the R function Im() with fetal fraction as the response variable, and i) fragment length profiles per 5 Mb or ii) 256 4 base-pair sequence motif frequencies as explanatory variables. A regression coefficient and a residual for each genomic bin is obtained as the result of the model:

$$Fetal Fraction in training samples = Coefficient \times fragment profile or \\ \\ motif frequencies in training samples + Residual$$

The Elastic net model was trained using the R package ‘glmnet’ with fetal fraction as the response variable, and i) fragment length profiles per 5 Mb or ii) 256 4 base-pair sequence motif frequencies as explanatory variables. Five-fold cross-validation was used for model training. The model combines L1 and L2 regularization for estimating regression coefficients.

The XGBoost model was trained using the R package “xgboost” with parameters “nrounds=100, max_depth=6, eta=0.3, gamma=0, colsample_bytree=1, min_child_weight=1, subsample=1”. Fetal fraction was set as the response variable, and i) fragment length profiles per 5 Mb or ii) 256 4 base-pair sequence motif frequencies were set as explanatory variables. XGBoost is a decision tree-based method. The training process determines a set of hyperparameters in the tree that best describes the data in the training dataset.

Techniques such as randomized parameter search and 5-fold cross-validation (CV) were used to prevent overfitting of the models.

Models trained as described above were then applied to the test data set to predict the fetal fraction for test samples using the R codes below:

model_linear_regression <- lm(FF ~ ., data = train)
model_elastic_net <- train(FF ~ ., data=train, method = “glmnet”,
tuneLength = 25, trControl = trControl)
model_xgboost <- train(FF ~ ., data=train, method = “xgbTree”,
tuneGrid = grid_default, trControl = trControl, verbosity = 0)

The trained linear regression model was applied to the test samples to estimate fetal fraction in test samples with an R predict() function:

$$Predicted Fetal Fraction = Coefficient from trained model \times fragment \\ \\ profile or motif frequencies in test samples + Residual from trained \\ \\ model$$

The trained elastic net model was applied to the test samples to estimate fetal fraction in test samples with coefficients and residuals estimated from training samples with an R predict() function.

The trained XGBoost model was applied to the test samples to estimate fetal fraction in test samples with hyperparameters estimated from training samples with an R predict() function.

The predicted fetal fraction was then compared with true fetal fraction to estimate model accuracy. Specifically, each model was evaluated on the test data set using two metrics: root mean squared error (RMSE) and correlation with truth fetal fraction. The metric RMSE was used to measure the model accuracy according to the following equation:

$$RMSE = \sqrt{\frac{{\sum }_{i = 1}^{N} {( {FF}_{predicted} - {FF}_{true} )}^{2}}{N}}$$

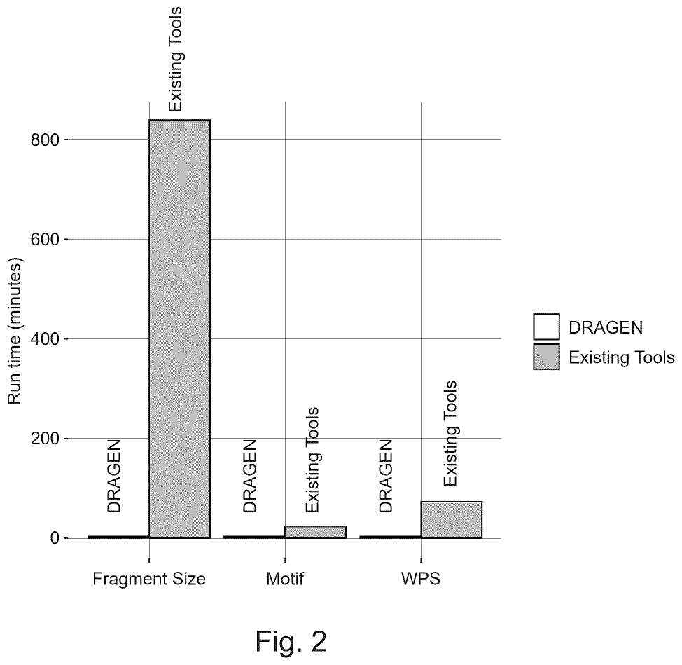

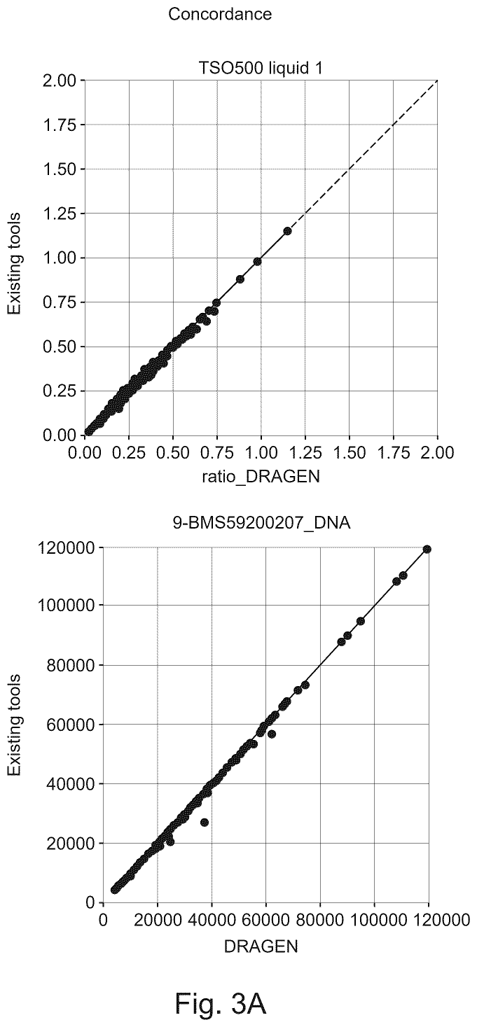

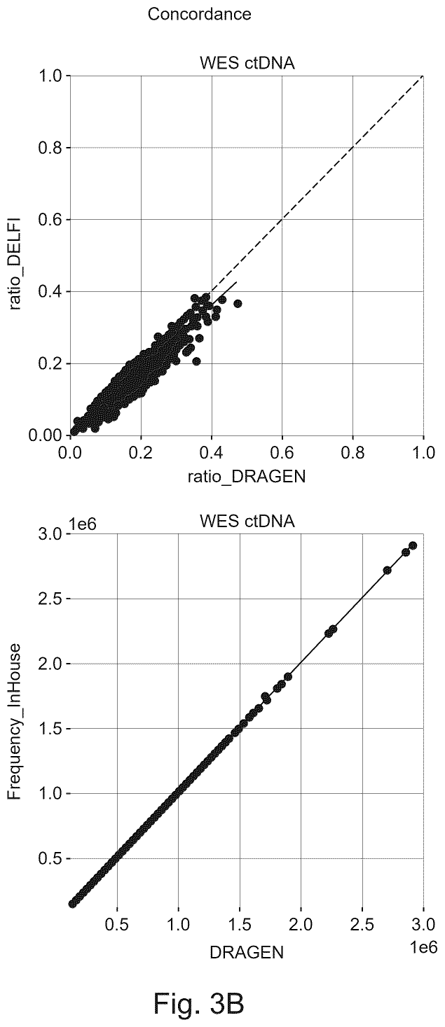

The method described in this Example for estimating fetal fraction in a test sample (DRAGEN) is at least two orders of magnitude faster than existing tools (see FIG. 2). FIG. 3 shows fragment profile (upper row) and sequence motif frequency (bottom row) from DRAGEN (x-axis) is highly concordant with existing tool (y-axis) in TSO500 and whole exome sequencing (WES) cfDNA datasets.

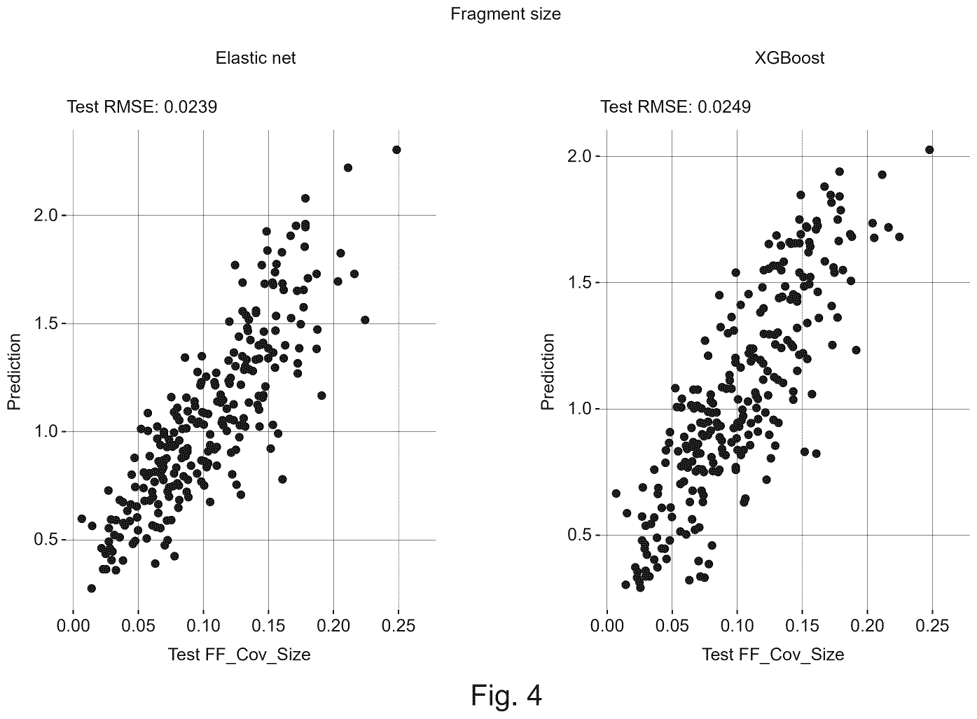

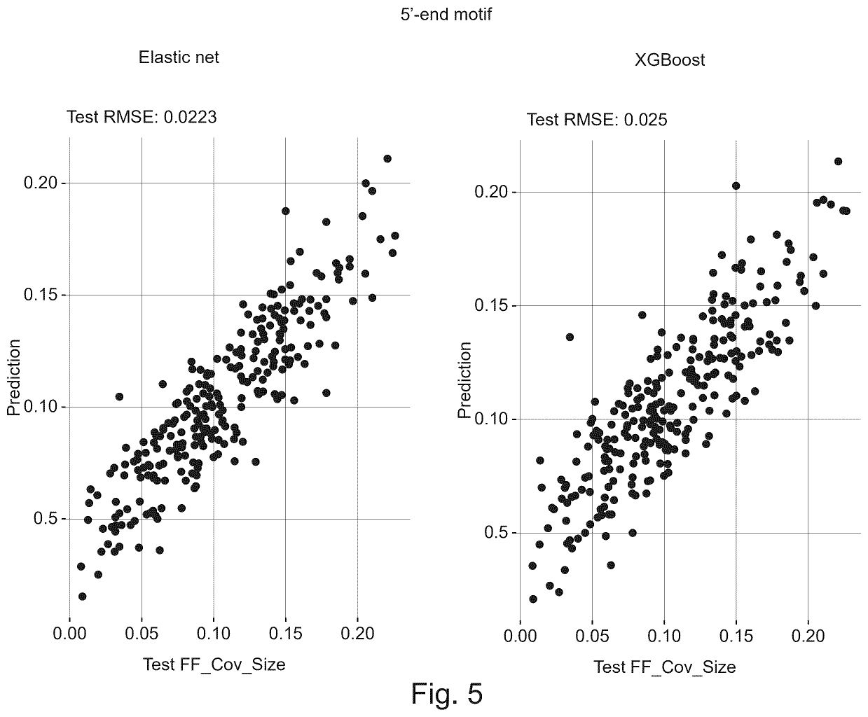

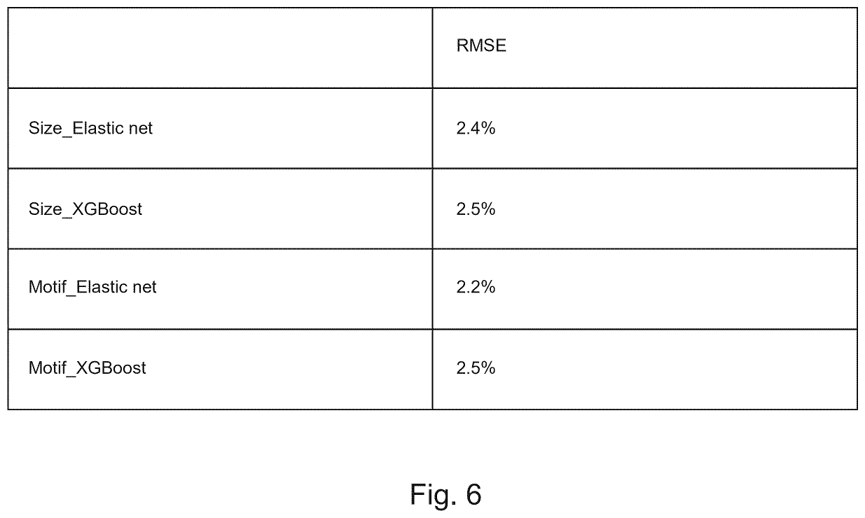

Models trained with only fragment size and Elastic net or XGBoost models are shown in FIG. 4. Fetal fraction predicted by model (y-axis) showed high consistency with truth data (x-axis). Models trained with only 5′-end sequence motif frequencies and Elastic net or XGBoost models are shown in FIG. 5. Fetal fraction predicted by model (y-axis) showed high consistency with truth data (x-axis). FIG. 6 provides a table showing a sequence motif-based method showed similar error rate of 2.5% when compared to a traditional fragment size-based method.

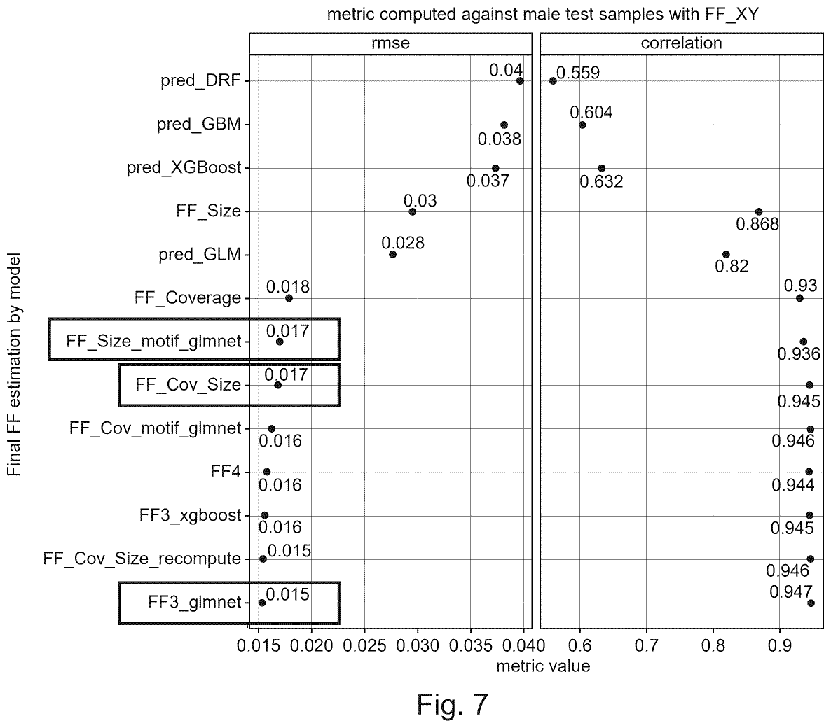

An overview of performance on a variety of models is shown in FIG. 7, which indicates combining sequence motif with fragment size or coverage features improves prediction accuracy. Coverage features generally refers to features derived from read coverage (i.e., the amount of mapped reads at given loci or portions of a genome) across a whole genome. RMSE (left) and correlation (right) of different models is provided. Y-axis from top to bottom are predictions from 1) distributed random forest (DRF) using only sequence motif; 2) gradient boosting (GBM) using only sequence motif; 3) XGBoost using only sequence motif; 4) FF_Size model from NIPT team; 5) Elastic net/Generalized linear model (GLM) using only sequence motif; 6) FF_Coverage model which utilizes genome-wide read coverage to predict fetal fraction (e.g., as described in U.S. Pat. No. 10,622,094 and in Kim et al. 2015 Prenatal Diagnosis 35:1-6, each of which are incorporated by reference in their entirety; 7) GLM model using both fragment size and sequence motif; 8) FF_Cov_Size model from NIPT team; 9) GLM model using fragment coverage and sequence motif; 10) FF4 model from NIPT team; 11) XGBoost model using fragment size, coverage, and sequence motif; 12) FF_Cov_Size_recompute model from NIPT team; 13) GLM model using fragment size, coverage, and sequence motif.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claim(s) that follow(s).

1. A method for estimating a fraction of fetal nucleic acid in a test sample from a pregnant subject comprising:
a) obtaining sequence reads mapped to a reference genome, wherein the sequence reads are reads of circulating cell-free (CCF) nucleic acid from a test sample from a pregnant subject;
b) measuring fragment lengths for a plurality of circulating cell-free nucleic acid fragments;
c) generating one or more fragment length profiles for the test sample;
d) determining a sequence motif for a plurality of circulating cell-free nucleic acid fragment ends;
e) determining one or more sequence motif frequencies for the test sample; and
f) estimating a fraction of fetal nucleic acid for the test sample according to i) the one or more fragment length profiles for the test sample, and ii) the one or more sequence motif frequencies for the test sample.

2. The method of claim 1, wherein:
i) the sequence reds are obtained by a paired-end sequencing process and the sequence reads are paired-end sequence reads;
ii) the fragment lengths are measured in (b) according to mapped positions of the paired-end sequence reads; and
iii) the fragment lengths are measured for a plurality of genomic intervals.

3. The method of claim 1, wherein the one or more fragment length profiles are generated in (c) according to ratios of X to Y for a plurality of genomic intervals, wherein X is the number of CCF nucleic acid fragments having a length within a first selected fragment length range, and Y is the number of CCF nucleic acid fragments having a length within a second selected fragment length range.

4. The method of claim 3, wherein the first selected fragment length range is about 80 bases to about 150 bases and the second selected fragment length range is about 151 bases to about 300 bases.

5. The method of claim 1, wherein the one or more fragment length profiles are generated in (c) for one or more genomic segments.

6. The method of claim 1, wherein:
i) the sequence motif in (d) is a 5′ sequence motif;
ii) the sequence motif in (d) is a four-base pair (bp) sequence motif; or
iii) the sequence motif in (d) is a 5′ sequence motif and a four-base pair (bp) sequence motif.

7. The method of claim 1, wherein (e) comprises determining one or more sequence motif frequencies for one or more chromosomes.

8. The method of claim 1, wherein (e) comprises determining one or more frequencies for one or more sequence motifs chosen from GGAA, AGAA, GTTT, GAAT, and GGTT.

9. The method of claim 1, wherein the one or more sequence motif frequencies are determined according to the frequencies of one or more sequence motifs in the mapped sequence reads for one or more chromosomes.

10. The method of claim 1, wherein estimating the fraction of fetal nucleic acid for the test sample in (f) comprises applying one or more model parameters from one or more models to i) the one or more fragment length profiles for the test sample, and ii) the one or more sequence motif frequencies for the test sample.

11. The method of claim 10, wherein the model parameters are obtained from a training set of samples, and wherein the fraction of fetal nucleic acid is known for each sample in the training set of samples.

12. The method of claim 11, wherein:
i) the one or more model parameters are obtained from the training set according to a fitted relation between 1) the fraction of fetal nucleic acid for each sample in the training set of samples and 2) one or more fragment length profiles for each sample in the training set of samples;
ii) the one or more model parameters are obtained from the training set according to a fitted relation between 1) the fraction of fetal nucleic acid for each sample in the training set of samples and 2) one or more sequence motif frequencies for each sample in the training set of samples; or
iii) the one or more model parameters are obtained from the training set according to a fitted relation between 1) the fraction of fetal nucleic acid for each sample in the training set of samples and 2) one or more fragment length profiles and one or more sequence motif frequencies for each sample in the training set of samples.

13. The method of claim 10, wherein the one or more model parameters comprise a coefficient derived from the one or more models.

14. The method of claim 10, wherein the one or more models comprise linear regression, and wherein estimating the fraction of fetal nucleic acid for the test sample in (f) comprises applying a regression coefficient from the linear regression to i) the one or more fragment length profiles for the test sample, and ii) the one or more sequence motif frequencies for the test sample.

15. The method of claim 10, wherein the one or more models comprise Elastic net, and wherein estimating the fraction of fetal nucleic acid for the test sample in (f) comprises applying a coefficient from the Elastic net model to i) the one or more fragment length profiles for the test sample, and ii) the one or more sequence motif frequencies for the test sample.

16. The method of claim 10, wherein the one or more models comprise XGBoost, and wherein estimating the fraction of fetal nucleic acid for the test sample in (f) comprises applying a coefficient from the XGBoost model to i) the one or more fragment length profiles for the test sample, and ii) the one or more sequence motif frequencies for the test sample.

17. The method of claim 1, further comprising prior to (a), sequencing the circulating cell-free (CCF) nucleic acid from the test sample by a sequencing process wherein:
i) the sequencing process is a non-targeted sequencing process;
ii) the sequencing process is a massively parallel sequencing process; or
iii) the sequencing process is a non-targeted massively parallel sequencing process.

18. A system comprising one or more microprocessors and memory, which memory comprises instructions executable by the one or more microprocessors and which memory comprises sequence reads mapped to a reference genome, wherein the sequence reads are reads of circulating cell-free (CCF) nucleic acid from a test sample from a pregnant subject, and wherein the instructions executable by the one or more microprocessors are configured to:
a) measure fragment lengths for a plurality of circulating cell-free nucleic acid fragments;
b) generate one or more fragment length profiles for the test sample;
c) determine a sequence motif for a plurality of circulating cell-free nucleic acid fragment ends;
d) determine one or more sequence motif frequencies for the test sample; and
e) estimate a fraction of fetal nucleic acid for the test sample according to i) the one or more fragment length profiles for the test sample, and ii) the one or more sequence motif frequencies for the test sample.

19. A machine comprising one or more microprocessors and memory, which memory comprises instructions executable by the one or more microprocessors and which memory comprises sequence reads mapped to a reference genome, wherein the sequence reads are reads of circulating cell-free nucleic acid from a test sample from a pregnant subject, and wherein the instructions executable by the one or more microprocessors are configured to:
a) measure fragment lengths for a plurality of circulating cell-free nucleic acid fragments;
b) generate one or more fragment length profiles for the test sample;
c) determine a sequence motif for a plurality of circulating cell-free nucleic acid fragment ends;
d) determine one or more sequence motif frequencies for the test sample; and
e) estimate a fraction of fetal nucleic acid for the test sample according to i) the one or more fragment length profiles for the test sample, and ii) the one or more sequence motif frequencies for the test sample.

20. (canceled)
